(19) United States
(12) Patent Application Publication
(10) Pub. No.: US 2010/0075966 A1
(43) Pub. Date: Mar. 25, 2010

(54) Substituted Dihydroimidazoles and their Use in the Treatment of Tumors

(75) Inventors: Sylvie Chamoin, Basel (CH); Hans- Jorg Roth, Basel (CH); Juerg Zimmermann, Basel (CH); Thomas Zoller, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/516,414

(22) PCT Filed: Nov. 26, 2007
(86) PCT No.: PCT/EP07/62804

§ 371 Date: May 27, 2009

(30) Foreign Application Priority Data
Nov. 27, 2006 (EP) 06124802.7

(51) Int. Cl. A61K 31/5377; C07D 233/54; A61K 31/4164; C07D 403/02; A61K 31/496; C07D 413/14; C07D 413/02; C07D 401/02; A61K 31/4439; A61P 35/00

(52) U.S. Cl. 514/235.8; 548/334.5; 514/400; 544/370; 514/254.05; 544/121; 544/139; 546/272.7; 514/341

(57) ABSTRACT

The invention relates to dihydroimidazoles of formula rac-(I),

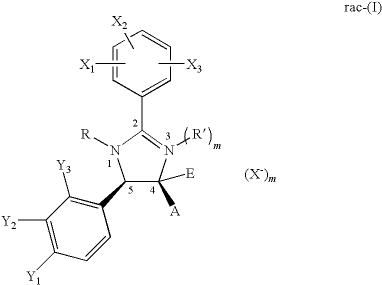

wherein the radicals and symbols are as defined herein; their use as inhibitors of the interaction of the MDM2 protein with a p53-like peptide, new pharmaceutical formulations comprising said compounds, said compounds for use in the therapeutic treatment of warm-blooded animals, especially humans, their use in the treatment of proliferative diseases or for the manufacture of pharmaceutical formulations useful in the treatment of proliferative diseases that respond to modulation of the interaction of the MDM2 protein with a p53-like peptide, a pharmaceutical formulation e.g. useful in the treatment of proliferative diseases that respond to modulation of the interaction of the MDM2 protein with a p53-like peptide comprising said compound, methods of treatment comprising administration of said compounds to a warm-blooded animal, and/or processes for the manufacture of said compounds.

The invention relates to substituted dihydroimidazoles, their use as inhibitors of the interaction of the MDM2 protein with a p53-like peptide, new pharmaceutical formulations comprising said compounds, said compounds for use in the therapeutic treatment of warm-blooded animals, especially humans, their use in the treatment of proliferative diseases and for the manufacture of pharmaceutical formulations useful in the treatment of proliferative diseases that respond to modulation of the interaction of the MDM2 protein with a p53-like peptide, a pharmaceutical formulation e.g. useful in the treatment of proliferative diseases that respond to modulation of the interaction of the MDM2 protein with a p53-like peptide comprising said compound, methods of treatment comprising administration of said compounds to a warm-blooded animal, and/or processes for the manufacture of said compounds.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in s the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This lo feedback control loop insures that both MDM2 and p53 are kept at a low level in normal proliferating cells. MDM2 is also a cofactor for E2F, which plays a central role in cell cycle regulation. The ratio of MDM2 to p53 (E2F) is dysregulated in many cancers. Frequently occurring molecular defects in the pI6INK4/pI9ARF locus, for instance, have been shown to affect MDM2 protein degradation. Inhibition of MDM2-p53 interaction in tumor cells with wild-type p53 should lead to accumulation of p53, cell cycle arrest and/or apoptosis.

MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might have effects in p53 mutant cells. Cis-2,4,5-Triphenyl-imidazolines being MDM2 antagonists are described in the prior art, e.g. in WO2003051359A1. It has now been found that the dihydroimidazoles of formula rac-(I) described herein are superior to known MDM2 antagonists with respect to their pharmacokinetic profile rendering the dihydroimidazoles of formula rac-(I) described herein better suitable for the development of pharmaceutical preparations for the treatment of proliferative diseases.

### DETAILED DESCRIPTION OF THE INVENTION

The invention especially relates to dihydroimidazoles of formula rac-(I),

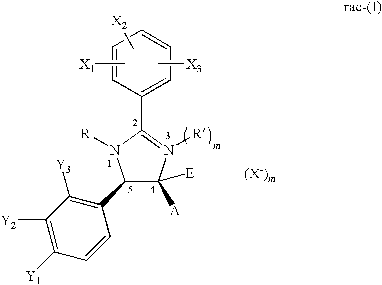

wherein

R is

(a) —C(O)R₁, wherein

R₁ denotes NRaRb, wherein Ra and Rb independently of each other denote hydrogen,
unsubstituted or substituted C1-C10 alkyl;
an unsubstituted or substituted monocyclic or bicyclic ring system comprising five to ten carbon atoms and being partially saturated or fully partially saturated; or
an unsubstituted or substituted monocyclic ring system being fully saturated or fully unsaturated and containing between two and five carbon atoms and one or two hetero atoms selected from N and O; or

wherein Ra and Rb together with the nitrogen atom to which they are attached represent an unsubstituted or substituted monocyclic ring system being fully saturated or partially saturated and containing between two and five carbon atoms and one or two hetero atoms selected from N and O; or

R₁ denotes unsubstituted or substituted C1-C8 alkyl, unsubstituted or substituted C3-C4-cycloalkyl, unsubstituted or substituted aryl or an unsubstituted or substituted mono- or bicyclic ring system being fully unsaturated, partially saturated or fully saturated and containing between two and eight carbon atoms and one, two or three hetero atoms selected from N, S and O;

(b) C1-C6 alkyl, which is unsubstituted or substituted by unsubstituted or substituted aryl or a mono- or bicyclic ring system being fully unsaturated and containing between two and six carbon atoms and one, two or three hetero atoms selected from N and O;

(c) C3-C5 alkenyl;

(d) —SO₂—R₆, wherein R₆ denotes unsubstituted or substituted C1-C4 alkyl, C3-C5 alkenyl, or unsubstituted or substituted aryl; or

(e) hydrogen;

R′ denotes C1-C6 alkyl,

m is 0 or 1 under the proviso that if m is 1, the nitrogen atom to which R′ is attached is positively charged,

X⁻ is an anion derived from an organic or inorganic acid,

X₁, X₂ and X₃ are independently selected from —OH, C1-C2 alkyl, C1-C6 alkoxy, Cl, Br, F, —CH₂OCH₃, and —CH₂OCH₂CH₃, or one of X₁, X₂ and X₃ is H and the other two are independently selected from hydroxy, C1-C6 alkyl, C1-C6 alkoxy, Cl, Br, F, CF₃, —CH₂OCH₃, —CH₂OCH₂CH₃, —OCH₂CH₂R₃, OCH₂CF₃, and —OR₄, or one of X₁, X₂ and X₃ is H and the other two taken together with the two carbon atoms to which they are attached form a 5- or 6-membered saturated ring that contains at least one hetero atom selected from S, N and O, wherein
R₃ is selected from F, —OCH₃, —N(CH₃)CH₃, Cl, Br, an unsaturated 5- and 6 membered ring containing at least one hetero atom wherein the hetero atom is selected from N and O, and
R₄ is a 3- to 5-membered saturated ring;

Y₁ denotes halo, NO₂, CN, or —CCH;

Y₂ denotes hydrogen or halo;

Y₃ denotes hydrogen or hydroxy;

A is alkyl, cycloalkyl being unsubstituted or substituted by alkyl, or a radical of subformula (Ia),

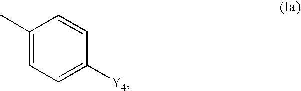

wherein Y₄ denotes halo, NO₂, CN, or —CCH; and

E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or

C(O)Z, wherein

Z represents

hydroxy, C1-C4 alkoxy, amino which is mono- or disubstituted by unsubstituted or substituted C1-C6 alkyl, or an unsubstituted or substituted mono- or bicyclic ring system being fully unsaturated, partially saturated or fully saturated and containing between two and eight carbon atoms and one, two or three hetero atoms selected from N, S and O, such ring system;

and to tautomers thereof,

and to salts of such dihydroimidazoles or such tautomer.

The invention pertains in particular to dihydroimidazole of formula rac-(I) wherein

R is

(a) —C(O)R₁, wherein

R₁ denotes NRaRb, wherein Ra and Rb independently of each other denote hydrogen,
unsubstituted or substituted C1-C10 alkyl;
an unsubstituted or substituted monocyclic or bicyclic ring system comprising five to ten carbon atoms and being partially saturated or fully partially saturated; or
an unsubstituted or substituted monocyclic ring system being fully saturated or fully unsaturated and containing between two and five carbon atoms and one or two hetero atoms selected from N and O; or

wherein Ra and Rb together with the nitrogen atom to which they are attached represent an unsubstituted or substituted monocyclic ring system being fully saturated or partially saturated and containing between two and five carbon atoms and one or two hetero atoms selected from N and O; or

R₁ denotes unsubstituted or substituted C1-C8 alkyl, unsubstituted or substituted C3-C4-cycloalkyl, unsubstituted or substituted aryl or an unsubstituted or substituted mono- or bicyclic ring system being fully unsaturated, partially saturated or fully saturated and containing between two and eight carbon atoms and one, two or three hetero atoms selected from N, S and O;

(b) C1-C4 alkyl, which is unsubstituted or substituted by unsubstituted or substituted aryl or a mono- or bicyclic ring system being fully unsaturated and containing between two and six carbon atoms and one, two or three hetero atoms selected from N and O;

(c) C3-C5 alkenyl;

(d) —SO₂—R₆, wherein R₆ denotes unsubstituted or substituted C1-C4 alkyl, C3-C5 alkenyl, or unsubstituted or substituted aryl; or

(e) hydrogen;

R′ denotes C1-C6 alkyl,

m is 0 or 1 under the proviso that if m is 1, the nitrogen atom to which R′ is attached is positively charged,

X⁻ is an anion derived from an organic or inorganic acid,

X₁, X₂ and X₃ are independently selected from —OH, C1-C2 alkyl, C1-C6 alkoxy, Cl, Br, F, —CH₂OCH₃, and —CH₂OCH₂CH₃, or one of X₁, X₂ and X₃ is H and the other two are independently selected from hydroxy, C1-C6 alkyl, C1-C6 alkoxy, Cl, Br, F, CF₃, —CH₂OCH₃, —CH₂OCH₂CH₃, —OCH₂CH₂R₃, OCH₂CF₃, and —OR₄, or one of X₁, X₂ and X₃ is H and the other two taken together with the two carbon atoms to which they are attached form a 5- or 6-membered saturated ring that contains at least one hetero atom selected from S, N and O, wherein
R₃ is selected from F, —OCH₃, —N(CH₃)CH₃, Cl, Br, an unsaturated 5- and 6 membered ring containing at least one hetero atom wherein the hetero atom is selected from N and O, and
R₄ is a 3- to 5-membered saturated ring;

Y₁ denotes halo, NO₂, CN, or —CCH;

Y₂ and Y₃ denote hydrogen;

A is a radical of subformula (Ia), wherein Y₄ denotes halo, NO₂, CN, or —CCH; and

E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or

C(O)Z, wherein

Z represents

hydroxy, C1-C4 alkoxy, amino which is mono- or disubstituted by unsubstituted or substituted C1-C6 alkyl, or an unsubstituted or substituted mono- or bicyclic ring system being fully unsaturated, partially saturated or fully saturated and containing between two and eight carbon atoms and one, two or three hetero atoms selected from N, S and O, such ring system.

More specifically, the present invention provides dihydroimidazoles of formula rac-(I),

wherein

R is

(a) —C(O)R₁, wherein

R₁ denotes NRaRb,

wherein Ra and Rb independently of each other denote
C1-C10 alkyl which is unsubstituted or mono-, di- or trisubstituted by cyano, C1-C4 alkoxy which is unsubstituted or substituted by phenyl, C3-C6 cycloalkyl, hydroxy, carbamoyl, N—C1-C4 alkyl carbamoyl, di(C1-C4 alkyl)-amino; a monocyclic or bicyclic ring system being unsaturated, partially saturated or fully saturated and containing between two and ten carbon atoms and one or two hetero atoms selected from N, O and S, such ring system being unsubstituted or substituted by C1-C4 alkyl, C2-C4 alkanoyl or oxo; or phenyl, which is unsubstituted or mono- or disubstituted by halo, aminosulfonyl, or a monocyclic ring system being fully saturated and containing between two and five carbon atoms and one or two hetero atoms selected from N and O, such ring system being unsubstituted or substituted by C1-C4 alkyl;
hydrogen,
a monocyclic or bicyclic ring system comprising five to ten carbon atoms and being partially saturated or fully partially saturated and optionally substituted by hydroxy, hydroxy C1-C4 alkyl or carbamoyl; or
a monocyclic ring system being fully saturated or fully unsaturated and containing between two and five carbon atoms and one or two hetero atoms selected from N and O, such ring system being unsubstituted or substituted by C1-C4 alkyl; or

wherein Ra and Rb together with the nitrogen atom to which they are attached represent a monocyclic ring system being fully saturated or partially saturated and containing between two and five carbon atoms and one or two hetero atoms selected from N and O, such ring system being unsubstituted or mono- or disubstituted by hydroxy, C2-C4 alkanoyl, carbamoyl, C1-C4 alkoxy carbonyl, C1-C4 alkyl which is unsubstituted or substituted by hydroxy, di(C1-C4 alkyl)-amino, morpholinyl carbonyl, piperidinyl carbonyl or pyrrolidinyl carbonyl; pyrimidinyl, phenyl, C1-C4 alkyl piperidinyl or oxo; phenyl, pyrrolidinyl, (1H)-2,3-dihydro-2-oxo-benzimidazolyl, or
R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, a mono- or bicyclic ring system being fully unsaturated or fully saturated and containing between two and eight carbon atoms and one, two or three hetero atoms selected from N, S and O, such ring system being unsubstituted or substituted by C1-C4 alkyl or oxo; or by phenyl which is substituted by di(C1-C4 alkyl)-amino;
C3-C4-cycloalkyl which is unsubstituted or substituted by C1-C4 alkyl;
phenyl being substituted by morpholinyl, di(C1-C4 alkyl)-amino or di(C1-C4 alkyl)-amino-sulfonyl; or;
a mono- or bicyclic ring system being fully unsaturated, partially saturated or fully saturated and containing between two and eight carbon atoms and one, two or three hetero atoms selected from N, S and O, such ring system being unsubstituted or substituted by halo, oxo, C1-C4 alkyl, thienyl-C1-C4 alkyl, halophenyl-C1-C4 alkyl, C1-C4 alkyl carbonyl or phenyl;

(b) C1-C4 alkyl, which is substituted by
phenyl which is mono- or di-substituted by C1-C4 alkyl, trifluoromethyl, C1-C4 alkoxy, trifluoromethoxy or cyano; or
a mono- or bicyclic ring system being fully unsaturated and containing between two and six carbon atoms and one, two or three hetero atoms selected from N and O;

(c) C3-C5 alkenyl;

(d) —SO₂—R₆, wherein R₆ denotes
C1-C4 alkyl, which is substituted by di(C1-C4 alkyl)-amino, a monocyclic ring system being fully saturated and containing between four and five carbon atoms and two hetero atoms selected from N and O, such ring system being substituted by C1-C4 alkyl, hydroxy C1-C4 alkyl or oxo;
C3-C5 alkenyl;
naphthyl; or
phenyl, which is substituted by halo; or

(e) hydrogen;

R′ denotes C1-C6 alkyl,

m is 0 or 1 under the proviso that if m is 1, the nitrogen atom to which R′ is attached is positively charged,

X⁻ is an anion derived from an organic or inorganic acid,

X₁, X₂ and X₃ are independently selected from —OH, C1-C2 alkyl, C1-C6 alkoxy, Cl, Br, F, —CH₂OCH₃, and —CH₂OCH₂CH₃, or one of X₁, X₂ and X₃ is H and the other two are independently selected from hydroxy, C1-C6 alkyl, C1-C6 alkoxy, Cl, Br, F, CF₃, —CH₂OCH₃, —CH₂OCH₂CH₃, —OCH₂CH₂R₃, OCH₂CF₃, and —OR₄, or one of X₁, X₂ and X₃ is H and the other two taken together with the two carbon atoms to which they are attached form a 5- or 6-membered saturated ring that contains at least one hetero atom selected from S, N and O, wherein
R₃ is selected from F, —OCH₃, —N(CH₃)CH₃, Cl, Br, an unsaturated 5- and 6 membered ring containing at least one hetero atom wherein the hetero atom is selected from N and O, and
R₄ is a 3- to 5-membered saturated ring;

Y₁ denotes Cl, Br, NO₂, CN, or —CCH;

Y₂ and Y₃ denote hydrogen;

A is a radical of subformula (Ia), wherein Y₄ denotes Cl, Br, NO₂, CN, or —CCH; and

E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or

C(O)Z, wherein

Z represents

hydroxy or C1-C4 alkoxy;

amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, a mono- or bicyclic ring system being fully unsaturated, partially saturated or fully saturated and containing between two and eight carbon atoms and one, two or three hetero atoms selected from N, S and O, such ring system being unsubstituted or substituted by C1-C4 alkyl, C1-C4 alkyl carbonyl and oxo; or phenyl, which is unsubstituted or substituted by C1-C4 alkyl piperazinyl, halo or amino sulfonyl;
C3-C5 alkinyl; 1,2-dihydro-indanyl, phenyl or C4-C6 cycloalkyl; or

a mono- or bicyclic ring system being fully unsaturated, partially saturated or fully saturated and containing between two and eight carbon atoms and one, two or three hetero atoms selected from N, S and O, such ring system being unsubstituted or substituted by C1-C4 alkyl, di(C1-C4 alkyl)-amino C1-C4 alkyl, C1-C4 alkyl carbonyl, C1-C4 alkyl piperidinyl, oxo, phenyl, pyrrolidinyl, amino carbonyl, C1-C4 alkoxy carbonyl or pyrimidyl;

or a tautomer thereof,

or a salt of such dihydroimidazole or its tautomer.

Preferred are dihydroimidazole of formula rac-(I), wherein

R is

(a) —C(O)R₁, wherein

R₁ denotes NRaRb,

wherein Ra and Rb independently of each other denote
C1-C10 alkyl which is unsubstituted or mono-, di- or trisubstituted by cyano, C1-C4 alkoxy which is unsubstituted or substituted by phenyl, C3-C6 cycloalkyl, hydroxy, carbamoyl, N-C1-C4 alkyl carbamoyl, di(C1-C4 alkyl)-amino; a monocyclic or bicyclic ring system being unsaturated, partially saturated or fully saturated and containing between two and ten carbon atoms and one or two hetero atoms selected from N, O and S, such ring system being unsubstituted or substituted by C1-C4 alkyl, C2-C4 alkanoyl or oxo; or phenyl, which is unsubstituted or mono- or disubstituted by halo, aminosulfonyl, or a monocyclic ring system being fully saturated and containing between two and five carbon atoms and one or two hetero atoms selected from N and O, such ring system being unsubstituted or substituted by C1-C4 alkyl;
hydrogen,
a monocyclic or bicyclic ring system comprising five to ten carbon atoms and being partially saturated or fully partially saturated and optionally substituted by hydroxy, hydroxy C1-C4 alkyl or carbamoyl; or
a monocyclic ring system being fully saturated or fully unsaturated and containing between two and five carbon atoms and one or two hetero atoms selected from N and O, such ring system being unsubstituted or substituted by C1-C4 alkyl; or

wherein Ra and Rb together with the nitrogen atom to which they are attached represent a monocyclic ring system being fully saturated or partially saturated and containing between two and five carbon atoms and one or two hetero atoms selected from N and O, such ring system being unsubstituted or mono- or disubstituted by hydroxy, C2-C4 alkanoyl, carbamoyl, C1-C4 alkoxy carbonyl, C1-C4 alkyl which is unsubstituted or substituted by hydroxy, di(C1-C4 alkyl)-amino, morpholinyl carbonyl, piperidinyl carbonyl or pyrrolidinyl carbonyl; pyrimidinyl, phenyl, C1-C4 alkyl piperidinyl or oxo; phenyl, pyrrolidinyl, (1H)-2,3-dihydro-2-oxo-benzimidazolyl, or
R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, a mono- or bicyclic ring system being fully unsaturated or fully saturated and containing between two and eight carbon atoms and one, two or three hetero atoms selected from N, S and O, such ring system being unsubstituted or substituted by C1-C4 alkyl or oxo; or by phenyl which is substituted by di(C1-C4 alkyl)-amino;
C3-C4-cycloalkyl which is unsubstituted or substituted by C1-C4 alkyl;
phenyl being substituted by morpholinyl, di(C1-C4 alkyl)-amino or di(C1-C4 alkyl)-amino-sulfonyl; or;
a mono- or bicyclic ring system being fully unsaturated, partially saturated or fully saturated and containing between two and eight carbon atoms and one, two or three hetero atoms selected from N, S and O, such ring system being unsubstituted or substituted by halo, oxo, C1-C4 alkyl, thienyl-C1-C4 alkyl, halophenyl-C1-C4 alkyl, C1-C4 alkyl carbonyl or phenyl;

(b) C1-C4 alkyl, which is substituted by
phenyl which is mono- or di-substituted by C1-C4 alkyl, trifluoromethyl, C1-C4 alkoxy, trifluoromethoxy or cyano; or
a mono- or bicyclic ring system being fully unsaturated and containing between two and six carbon atoms and one, two or three hetero atoms selected from N and O;

(c) C3-C5 alkenyl;

(d) —SO₂—R₆, wherein R₆ denotes
C1-C4 alkyl, which is substituted by di(C1-C4 alkyl)-amino, a monocyclic ring system being fully saturated and containing between four and five carbon atoms and two hetero atoms selected from N and O, such ring system being substituted by C1-C4 alkyl, hydroxy C1-C4 alkyl or oxo;
C3-C5 alkenyl;
naphthyl; or
phenyl, which is substituted by halo; or

(e) hydrogen;

R′ denotes C1-C6 alkyl,

m is 0 or 1 under the proviso that if m is 1, the nitrogen atom to which R′ is attached is positively charged,

X⁻ is an anion derived from an organic or inorganic acid,

X₁, X₂ and X₃ are independently selected from hydrogen and C1-C4 alkoxy,

Y₁ denotes halo;

Y₂ and Y₃ denote hydrogen;

A is a radical of subformula (Ia), wherein Y₄ denotes halo; and

E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or C(O)Z, wherein

Z represents

C1-C4 alkoxy;

amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, a mono- or bicyclic ring system being fully unsaturated, partially saturated or fully saturated and containing between two and eight carbon atoms and one, two or three hetero atoms selected from N, S and O, such ring system being unsubstituted or substituted by C1-C4 alkyl, C1-C4 alkyl carbonyl and oxo; or phenyl, which is unsubstituted or substituted by C1-C4 alkyl piperazinyl, halo or amino sulfonyl;
C3-C5 alkinyl; 1,2-dihydro-indanyl, phenyl or C4-C6 cycloalkyl; or

a mono- or bicyclic ring system being fully unsaturated, partially saturated or fully saturated and containing between two and eight carbon atoms and one, two or three hetero atoms selected from N, S and O, such ring system being unsubstituted or substituted by C1-C4 alkyl, di(C1-C4 alkyl)-amino C1-C4 alkyl, C1-C4 alkyl carbonyl, C1-C4 alkyl piperidinyl, oxo, phenyl, pyrrolidinyl, amino carbonyl, C1-C4 alkoxy carbonyl or pyrimidyl.

More preferred are dihydroimidazole of formula rac-(I), wherein

R is

(a) —C(O)R₁, wherein

R₁ denotes NRaRb,

wherein Ra and Rb independently of each other denote
C1-C10 alkyl which is unsubstituted or mono-, di- or trisubstituted by cyano, C1-C4 alkoxy which is unsubstituted or substituted by phenyl, hydroxy, carbamoyl, N—C1-C4 alkyl carbamoyl, di(C1-C4 alkyl)-amino, pyridyl, pyrrolyl, C1-C4 alkyl imidazolyl, furyl, indolyl, isochromanyl, benzothienyl; phenyl, which is unsubstituted or mono- or disubstituted by halo, morpholinyl, piperidinyl, aminosulfonyl, or C1-C4 alkyl piperazinyl; C3-C6 cycloalkyl; morpholinyl, tetrahydrofuranyl, tetrahydropyranyl, benzo[1,3]dioxolyl, C1-C4 alkyl piperazinyl, pyrrolidinyl which is unsubstituted or substituted by C1-C4 alkyl or oxo; C1-C4 alkyl-pyrazinyl, or piperidinyl, which is unsubstituted or substituted by C1-C4 alkyl or C2-C4 alkanoyl;
C5-C6 cycloalkyl which is unsubstituted or substituted by hydroxy, hydroxy C1-C4 alkyl or carbamoyl;
hydrogen,
indanyl, 2,3-dihydro-hydroxy-indanyl, 2,3-dihydro-2-indanyl, C1-C4 alkyl- pyrazolyl or
piperidinyl substituted by C1-C4 alkyl; or

wherein Ra and Rb together with the nitrogen atom to which they are attached represent piperazine being unsubstituted or mono- or disubstituted by C2-C4 alkanoyl, C1-C4 alkyl which is unsubstituted or substituted by hydroxy, di(C1-C4 alkyl)-amino, morpholinyl carbonyl, piperidinyl carbonyl or pyrrolidinyl carbonyl; pyrimidinyl, phenyl, C1-C4 alkyl piperidinyl or oxo;
pyrrolidine being substituted by hydroxy C1-C4 alkyl,
piperidine being unsubstituted or substituted by phenyl, benzyl, pyrrolidinyl, (1H)-2,3-dihydro-2-oxo-benzimidazolyl, carbamoyl, C1-C4 alkoxy carbonyl, hydroxy or hydroxy C1-C4 alkyl;
tetrahydro-pyrimidine;
C1-C4 alkyl-1,4-diaza-cycloheptane, phenyl C1-C4 alkyl-1,4-diaza-cycloheptane, or morpholine; or

R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, imidazolyl, C1-C4 alkyl-imidazolyl, pyrazolyl, piperidinyl, pyrrolidinyl, 1,1-dioxo-thiomorpholinyl, pyridyl, indolyl, N—C1-C4 alkyl-indolyl; or phenyl which is substituted by di(C1-C4 alkyl)-amino;
C3-C4-cycloalkyl which is unsubstituted or substituted by C1-C4 alkyl; phenyl being substituted by morpholinyl, di(C1-C4 alkyl)-amino or di(C1-C4 alkyl)-amino-sulfonyl;
N—C1-C4 alkyl-indolyl; N—C1-C4 alkyl-imidazolyl; quinoxalinyl; pyrazolo[1,5-a]pyrimidinyl being mono- or disubstituted by C1-C4 alkyl; [1,6]naphthyridinyl; oxazolyl being substituted by phenyl or C1-C4 alkyl; pyrazolyl being mono-, di- or trisubstituted by C1-C4 alkyl and halo;
piperidinyl, being unsubstituted or substituted by C1-C4 alkyl or C1-C4 alkyl carbonyl; tetrahydropyrimidyl, disubstituted by oxo, or pyrrolidinyl which is mono- or disubstituted by radicals independently selected from the group consisting of C1-C4 alkyl, thienyl-C1-C4 alkyl, chlorophenyl-C1-C4 alkyl and oxo;

(b) C1-C4 alkyl, which is substituted by phenyl which is mono- or di-substituted by C1-C4 alkyl, trifluoromethyl, C1-C4 alkoxy, trifluoromethoxy or cyano;
pyridyl; or
benzo[c]-1-oxa-2,5-diazolyl;
(c) C3-C5 alkenyl;
(d) —SO₂—R₆, wherein R₆ denotes
C1-C4 alkyl, which is substituted by di(C1-C4 alkyl)-amino, morpholinyl or piperazinyl which is further substituted by C1-C4 alkyl, hydroxy C1-C4 alkyl or oxo;
C3-C5 alkenyl;
naphthyl; or
phenyl, which is substituted by halo; or

(e) hydrogen;

R′ denotes C1-C6 alkyl,

m is 0 or 1 under the proviso that if m is 1, the nitrogen atom to which R′ is attached is positively charged,

X⁻ is an anion derived from an organic or inorganic acid,

X₁, X₂ and X₃ are independently selected from hydrogen and C1-C4 alkoxy,

Y₁ denotes halo;

Y₂ and Y₃ denote hydrogen;

A is a radical of subformula (Ia), wherein Y₄ denotes halo; and

E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or C(O)Z, wherein

Z represents

C1-C4 alkoxy;

amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, pyridyl, pyrrolyl, imidazolyl, C1-C4 alkyl imidazolyl, C1-C4 alkyl pyrimidyl, C1-C4 alkyl pyrazinyl, furyl, dihydroisochromanyl, tetrahydro-pyranyl, tetrahydrofuryl, morpholinyl, pyrrolidinyl, C1-C4 alkyl pyrrolidinyl, 2-oxo-pyrrolidinyl, piperidinyl, C1-C4 alkyl carbonyl piperidinyl, C1-C4 alkyl piperidinyl, C1-C4 alkyl piperazinyl; or phenyl, which is unsubstituted or substituted by C1-C4 alkyl piperazinyl, halo or amino sulfonyl; or C3-C5 alkinyl; pyridyl, thiazolyl, C1-C4 alkyl oxazolyl, isoxazolyl, pyrazinyl, pyrimidyl, C1-C4 alkyl pyrazolyl, 1,2-dihydro-indanyl, phenyl or C4-C6 cycloalkyl;

pyrrolidinyl;

piperazinyl, which is unsubstituted or substituted by C1-C4 alkyl, di(C1-C4 alkyl)-amino C1-C4 alkyl, C1-C4 alkyl carbonyl, C1-C4 alkyl piperidinyl, oxo or pyrimidyl;

piperidinyl, which is unsubstituted or substituted by phenyl, pyrrolidinyl, amino carbonyl or C1-C4 alkoxy carbonyl;

morpholinyl;

tetrahydrothiazolyl; or

C1-C4 alkyl-1,4-diaza-cycloheptane.

Highly preferred are furthermore dihydroimidazole of formula rac-(I), wherein

R is

(a) —C(O)R₁, wherein

R₁ denotes NRaRb,

wherein Ra and Rb independently of each other denote
C1-C10 alkyl which is unsubstituted or mono-, di- or trisubstituted by cyano, C1-C4 alkoxy which is unsubstituted or substituted by phenyl, hydroxy, carbamoyl, N-methyl carbamoyl, di(C1-C4 alkyl)-amino, pyridyl, 1-pyrrolyl, C1-C4 alkyl imidazolyl, furyl, 3-indolyl, isochromanyl, benzothienyl; phenyl, which is unsubstituted or mono- or disubstituted by chloro, morpholinyl, piperidinyl, aminosulfonyl, or C1-C4 alkyl piperazinyl; C3-C6 cycloalkyl; morpholin-4-yl, tetrahydrofuranyl, tetrahydropyranyl, benzo[1,3]dioxolyl, C1-C4 alkyl piperazinyl, pyrrolidinyl which is unsubstituted or substituted by C1-C4 alkyl or oxo; C1-C4 alkyl-pyrazinyl, or piperidinyl, which is unsubstituted or substituted by C1-C4 alkyl or C2-C4 alkanoyl;
C5-C6 cycloalkyl which is unsubstituted or substituted by hydroxy, hydroxy C1-C4 alkyl or carbamoyl;
hydrogen,
indanyl, 2,3-dihydro-2-hydroxy-indanyl, 2,3-dihydro-2-indanyl,
1-C1-C4 alkyl-pyrazolyl or
4-piperidinyl substituted by C1-C4 alkyl; or

wherein Ra and Rb together with the nitrogen atom to which they are attached represent piperazine being unsubstituted or mono- or disubstituted by C2-C4 alkanoyl, C1-C4 alkyl which is unsubstituted or substituted by hydroxy, di(C1-C4 alkyl)-amino, morpholinyl carbonyl, piperidinyl carbonyl or 1-pyrrolidinyl carbonyl; 2-pyrimidinyl, phenyl, C1-C4 alkyl piperidinyl or oxo;
pyrrolidine being substituted by hydroxy C1-C4 alkyl,
piperidine being unsubstituted or substituted by phenyl, benzyl, 1-pyrrolidinyl, (1H)-2,3-dihydro-2-oxo-benzimidazol-1-yl, carbamoyl, C1-C4 alkoxy carbonyl, hydroxy or hydroxy C1-C4 alkyl;
tetrahydro-pyrimidine;
4-C1-C4 alkyl-1,4-diaza-cycloheptane, 4-benzyl-1,4-diaza-cycloheptane, or morpholine; or

R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, imidazolyl, C1-C4 alkyl-imidazolyl, pyrazolyl, piperidinyl, pyrrolidinyl, 1,1-dioxo-thiomorpholinyl, pyridyl, indolyl, N—C1-C4 alkyl-indolyl; or phenyl which is substituted by di(C1-C4 alkyl)-amino;
C3-C4-cycloalkyl which is unsubstituted or substituted by C1-C4 alkyl;
phenyl being substituted by morpholinyl, di(C1-C4 alkyl)-amino or di(C1-C4 alkyl)-amino-sulfonyl;
N—C1-C4 alkyl-indolyl; N—C1-C4 alkyl-imidazolyl; quinoxalinyl; pyrazolo[1,5-a]pyrimidinyl being disubstituted by C1-C4 alkyl; [1,6]naphthyridinyl; oxazolyl being substituted by phenyl or C1-C4 alkyl; pyrazolyl being di- or trisubstituted by C1-C4 alkyl and chloro; piperidinyl, being unsubstituted or substituted by C1-C4 alkyl or C1-C4 alkyl carbonyl; tetrahydropyrimidyl, disubstituted by oxo, or pyrrolidinyl which is mono- or disubstituted by radicals independently selected from the group consisting of C1-C4 alkyl, thienyl-C1-C4 alkyl, chlorophenyl-C1-C4 alkyl and oxo;

(b) C1-C4 alkyl, which is substituted by
phenyl which is mono- or di-substituted by C1-C4 alkyl, trifluoromethyl, C1-C4 alkoxy, trifluoromethoxy or cyano;
pyridyl; or
benzo[c]-1-oxa-2,5-diazolyl;

(c) C3-C4 alkenyl;

(d) SO₂—R₆, wherein R₆ denotes
C1-C4 alkyl, which is substituted by di(C1-C4 alkyl)-amino, morpholinyl or piperazinyl which is further substituted by C1-C4 alkyl, hydroxy C1-C4 alkyl or oxo;
C3-C4 alkenyl;
naphthyl; or
phenyl, which is substituted by fluoro; or

(e) hydrogen;

R′ denotes C1-C6 alkyl,

m is 0 or 1 under the proviso that if m is 1, the nitrogen atom to which R′ is attached is positively charged,

X⁻ is an anion derived from an organic or inorganic acid,

X₁, X₂ and X₃ are independently selected from hydrogen and C1-C4 alkoxy,

Y₁ denotes halo;

Y₂ and Y₃ denote hydrogen;

A is a radical of subformula (Ia), wherein Y₄ denotes halo; and

E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl;

or C(O)Z, wherein

Z represents

C1-C4 alkoxy; C1-C4 alkyl;

amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, pyridyl, pyrrolyl, imidazolyl, C1-C4 alkyl imidazolyl, C1-C4 alkyl pyrimidyl, C1-C4 alkyl pyrazinyl, furyl, dihydro-isochromanyl, tetrahydro-pyranyl, tetrahydrofuryl, morpholinyl, pyrrolidinyl, C1-C4 alkyl pyrrolidinyl, 2-oxo-pyrrolidinyl, piperidinyl, C1-C4 alkyl carbonyl piperidinyl, C1-C4 alkyl piperidinyl, C1-C4 alkyl piperazinyl; or phenyl, which is unsubstituted or substituted by C1-C4 alkyl piperazinyl, fluoro or amino sulfonyl; or C3-C5 alkinyl; pyridyl, thiazolyl, C1-C4 alkyl oxazolyl, isoxazolyl, pyrazinyl, pyrimidyl, C1-C4 alkyl pyrazolyl, 1,2-dihydro-indanyl, phenyl or C4-C6 cycloalkyl;

pyrrolidinyl;

piperazinyl, which is unsubstituted or substituted by C1-C4 alkyl, di(C1-C4 alkyl)-amino C1-C4 alkyl, C1-C4 alkyl carbonyl, C1-C4 alkyl piperidinyl, oxo or pyrimidyl;

piperidinyl, which is unsubstituted or substituted by phenyl, pyrrolidinyl, amino carbonyl or C1-C4 alkoxy carbonyl;

morpholinyl;

tetrahydrothiazolyl; or

4-C1-C4 alkyl-1,4-diaza-cycloheptane;

or a tautomer thereof,

or a salt of such dihydroimidazole or its tautomer.

Particular preference is given to the compounds described in the Examples below.

In addition to the foregoing, in formula rac-(I) the following significances are preferred independently, collectively or in any combination or sub-combination:
1. The absolute configuration of C-5 in the dihydroimidazole ring of the dihydroimidazole of formula rac-(I) is “R”,
2. E denotes C(O)Z, wherein Z has the meanings as defined herein,
3. Z represents
C1-C4 alkoxy;
amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, pyridyl, pyrrolyl, imidazolyl, C1-C4 alkyl imidazolyl, C1-C4 alkyl pyrimidyl, C1-C4 alkyl pyrazinyl, furyl, dihydro-isochromanyl, tetrahydro-pyranyl, tetrahydrofuryl, morpholinyl, pyrrolidinyl, C1-C4 alkyl pyrrolidinyl, 2-oxo-pyrrolidinyl, piperidinyl, C1-C4 alkyl carbonyl piperidinyl, C1-C4 alkyl piperidinyl, C1-C4 alkyl piperazinyl; or phenyl, which is unsubstituted or substituted by C1-C4 alkyl piperazinyl, fluoro or amino sulfonyl; or C3-C5 alkinyl; pyridyl, thiazolyl, C1-C4 alkyl oxazolyl, isoxazolyl, pyrazinyl, pyrimidyl, C1-C4 alkyl pyrazolyl, 1,2-dihydro-indanyl, phenyl or C4-C6 cycloalkyl;
pyrrolidinyl;
piperazinyl, which is unsubstituted or substituted by C1-C4 alkyl, di(C1-C4 alkyl)-amino C1-C4 alkyl, C1-C4 alkyl carbonyl, C1-C4 alkyl piperidinyl, oxo or pyrimidyl;
piperidinyl, which is unsubstituted or substituted by phenyl, pyrrolidinyl, amino carbonyl or C1-C4 alkoxy carbonyl;
morpholinyl;
tetrahydrothiazolyl; or
4-C1-C4 alkyl-1,4-diaza-cycloheptane.

The general terms used hereinbefore and hereinafter preferably have the following meanings within the context of this disclosure, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms may be present in the (R)-, (S)- or (R,S)-configuration, preferably in the (R)- or (S)-configuration. The compounds may thus be present as mixtures of isomers or as pure isomers, preferably as enantiomer-pure diastereomers.

The invention relates also to possible tautomers of the dihydroimidazoles of formula rac-(I).

Halo or halogen is preferably fluoro, chloro, bromo or iodo, most preferably fluoro, chloro or bromo, if not defined otherwise.

Alkyl is preferably alkyl with from and including 1 up to and including 10, preferably from and including 1 to and including 8 carbon atoms, and is linear or branched; preferably, alkyl is methyl, ethyl, 1,1-dimethyl-ethyl, propyl, such as n-propyl or isopropyl, butyl, such as n-butyl, sec-butyl, isobutyl, tert-butyl or 1,5-dimethyl-hexyl.

Unsubstituted or substituted alkyl denotes alkyl as defined above which is preferably unsubstituted or mono-, di- or trisubstituted by unsubstituted or substituted aryl, cyano, C1-C4 alkoxy which is unsubstituted or substituted by phenyl, hydroxy, carbamoyl, N-methyl carbamoyl, amino, C1-C4 alkyl carbonyl amino, C1-C4 alkyl-phenyl carbonyl amino, di(C1-C4 alkyl)-amino, pyridyl, 1-pyrrolyl, imidazolyl, C1-C4 alkyl imidazolyl, pyrazolyl, furyl, indolyl, N—C1-C4 alkyl-indolyl, isochromanyl, benzothienyl; phenyl, which is unsubstituted or mono- or disubstituted by chloro, di(C1-C4 alkyl)-amino, morpholinyl, piperidinyl, aminosulfonyl, or C1-C4 alkyl piperazinyl; C3-C6 cycloalkyl; morpholin-4-yl, 1,1-dioxo-thiomorpholinyl, tetrahydrofuranyl, tetrahydropyranyl, benzo[1,3]dioxolyl, C1-C4 alkyl piperazinyl, pyrrolidinyl which is unsubstituted or substituted by C1-C4 alkyl or oxo; C1-C4 alkyl-pyrazinyl; piperidinyl, which is unsubstituted or substituted by C1-C4 alkyl or C2-C4 alkanoyl; or a mono- or bicyclic ring system being fully unsaturated and containing between two and six carbon atoms and one, two or three hetero atoms selected from N and O.

Alkoxy is preferably C1-C6 alkoxy, especially methoxy and isopropoxy.

Hydroxyalkyl is especially hydroxy-C1-C4 alkyl, preferably hydroxymethyl, 2-hydroxyethyl or 2-hydroxy-2-propyl.

Alkenyl is preferably C2-C5 alkenyl, more preferably C3-C5 alkenyl, and means in particular 2-propenyl or 2-butenyl.

Alkanoyl is preferably formyl or C1-C4 alkyl carbonyl, in particular acetyl.

Unsubstituted or substituted C3-C4-cycloalkyl means in particular cyclopropyl or cyclobutyl which is unsubstituted or substituted by C1-C4 alkyl.

Aryl can be unsubstituted or substituted and in particular means phenyl being unsubstituted or mono- or di-substituted by C1-C4 alkyl, trifluoromethyl, C1-C4 alkoxy, trifluoromethoxy, cyano, morpholinyl, di(C1-C4 alkyl)-amino or di(C1-C4 alkyl)-amino-sulfonyl.

Unsubstituted or substituted monocyclic or bicyclic ring system comprising five to ten carbon atoms and being partially saturated or fully saturated means in particular monocyclic C5-C6 cycloalkyl which preferably is unsubstituted or substituted by hydroxy, hydroxy C1-C4 alkyl or carbamoyl; indanyl, 2,3-dihydro-2-hydroxy-indanyl, or 2,3-dihydro-2-indanyl.

An unsubstituted or substituted monocyclic ring system being fully or partially saturated or fully unsaturated and containing between two and five carbon atoms and one or two hetero atoms selected from N and O means in particular piperazine being unsubstituted or mono- or disubstituted by C2-C4 alkanoyl, C1-C4 alkyl which is unsubstituted or substituted by hydroxy, di(C1-C4 alkyl)-amino, morpholinyl carbonyl, piperidinyl carbonyl or 1-pyrrolidinyl carbonyl; 2-pyrimidinyl, phenyl, C1-C4 alkyl piperidinyl or oxo; pyrrolidine being substituted by hydroxy C1-C4 alkyl, piperidine being unsubstituted or substituted by phenyl, benzyl, 1-pyrrolidinyl, (1H)-2,3-dihydro-2-oxo-benzimidazol-1-yl, carbamoyl, C1-C4 alkoxy carbonyl, hydroxy or hydroxy C1-C4 alkyl; tetrahydro-pyrimidine; 4-C1-C4 alkyl-1,4-diaza-cycloheptane, 4-benzyl-1,4-diaza-cycloheptane, morpholine or 1-C1-C4 alkyl-pyrazolyl.

Unsubstituted or substituted mono- or bicyclic ring system being fully unsaturated, partially saturated or fully saturated and containing between two and eight carbon atoms and one, two or three hetero atoms selected from N, S and O means in particular N—C1-C4 alkyl-indolyl; N—C1-C4 alkyl-imidazolyl; quinoxalinyl; pyrazolo[1,5-a]pyrimidinyl being disubstituted by C1-C4 alkyl; [1,6]naphthyridinyl; oxazolyl being substituted by phenyl or C1-C4 alkyl; pyrazolyl being di- or trisubstituted by C1-C4 alkyl and chloro; piperidinyl, being unsubstituted or substituted by C1-C4 alkyl, C1-C4 alkyl carbonyl, phenyl, pyrrolidinyl, amino carbonyl or C1-C4 alkoxy carbonyl; 4-C1-C4 alkyl-1,4-diaza-cycloheptanyl; tetrahydrothiazolyl; tetrahydropyrimidyl, disubstituted by oxo, pyrrolidinyl which is unsubstituted or mono- or disubstituted by radicals independently selected from the group consisting of C1-C4 alkyl, thienyl-C1-C4 alkyl, chlorophenyl-C1-C4 alkyl and oxo; morpholinyl; piperazinyl, which is unsubstituted or substituted by C1-C4 alkyl, di(C1-C4 alkyl)-amino C1-C4 alkyl, C1-C4 alkyl carbonyl, C1-C4 alkyl piperidinyl, oxo or pyrimidyl;

Unsubstituted or substituted mono- or bicyclic ring system being fully unsaturated and containing between two and six carbon atoms and one, two or three hetero atoms selected from N and O means in particular benzo[c]-1-oxa-2,5-diazolyl.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

Such salts are formed, for example, as acid addition salts, preferably with organic or inorganic acids, from the dihydroimidazoles of formula rac-(I) with a basic nitrogen atom, especially the pharmaceutically acceptable salts. Suitable inorganic acids are, for example, halogen acids, such as hydrochloric acid, sulfuric acid, or phosphoric acid.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

The dihydroimidazoles of formula rac-(I) have valuable pharmacological properties, as described hereinbefore and hereinafter.

The compounds according to the present invention show strong anti-tumor activity against various tumor cell lines. This anti-tumor activity indicates that “compounds of the present invention and pharmaceutically acceptable salts thereof can be anti-tumor agents.

The ability of the dihydroimidazoles of formula rac-(I) to inhibit the interaction between p53 and MDM2 proteins can be measured e.g. by an ELISA (Enzyme-Linked Immuno Sorbent Assay) in which recombinant GST-tagged MDM2 binds to a peptide that resembles the MDM2-interacting region of p53 (Bottger et al., J. Mol. Bio. 1997, Vol. 269, pas. 744-756). This peptide is immobilized to the surface of a 96 well plate via N-terminal biotin which binds to streptavidin-coated wells. MDM2 is added to each well in the presence of anti-MDM2 mouse monoclonal antibody (SMP-14, Santa Cruz Biotech). After removal of the unbound MDM2 protein, a peroxydase-linked secondary antibody (anti-mouse IgG, Roche Molecular Biochemicals) and the amount of peptide-bound MDM2 is determined calorimetrically by the addition of a peroxydase substrate (MTB Microwell Peroxydase Substrate System, Kirkegaard & Perry Labs).

Test plates are prepared by coating with streptavidin (5 mg/ml in PBS) for 2 hours followed by a PBS (phosphate-buffered saline) wash and overnight blocking with 150 ml of blocking buffer containing 2 mg/ml bovine serum albumin (Sigma) and 0.05% Tween 20 (Sigma) in PBS at 4° C. Biotinylated peptide (1 mM) is added to each well in 50 ml of blocking buffer and washed extensively after 1 h incubation. Test compounds are diluted in a separate 96 well plate and added in triplicate to a compound incubation plate containing a mix of the MDM2 protein and anti-MDM2 antibody. After 20 min incubation, the content of the plate is transferred to the test plate and incubated for an additional 1 hour. The secondary anti-mouse IgG antibody is added to the test plate preceeded and followed by a triple wash with 0.05% Tween 20 in PBS. Finally, peroxydase substrate is added to each well and the absorption is read using a plate reader (MR7000, Dynatech) at 450 nm. The inhibitory activity of the test compounds is measured as a percentage of the bound MDM2 in treated vs. untreated wells and IC50 is calculated.

In assays Ike the one described above, compounds of the present invention as exemplified show IC₅₀s from about 70 nM to about 2 mM.

On the basis of these studies, a dihydroimidazole of formula rac-(I) shows therapeutic efficacy especially against proliferative diseases. Preferably, the proliferative disorder is cancer and most preferably the cancer is breast, colon, lung or prostate cancer.

Pharmacokinetic data can be obtained be the test described in the following:

The dihydroimidazole of formula rac-(I) to be tested is formulated for administration to female OF1 mice from IFACREDO, France, by first dissolving in N-methyl-pyrrolidone (NMP), and then by diluting with PEG300 to a final concentration of 10% v/v NMP: 90% v/v PEG300, producing a clear solution of the compound. The concentrations are adjusted to deliver a constant volume of 10 mL/kg body weight. The compound is prepared immediately before use. The formulated compound is administered perorally by gavage to provide dosages of 50 mg/kg. At the allotted time points mice (4 at each time) are anesthetized with 3% isoflurane in medical oxygen and blood samples are obtained by heart puncture into heparinized tubes (ca. 30 IU/mL). The animals are subsequently killed without recovering from the anesthetic. Plasma is prepared from the blood by centrifugation (10,000 g, 5 min) and either analyzed immediately or stored frozen at −70° C.

The plasma samples (10-250 μL) are e.g. spiked with 5 μL of internal standard, mixed with 200 μL 0.1 M NaOH and 500 μL Chloroform in a 1.5 mL Eppendorf tube and shaken vigorously for 10 minutes on an Eppendorf mixer. Thereafter, the mixture is centrifuged (3 min at 10′000×g), the organic phase transferred to a second Eppendorf tube and evaporated to dryness in a vacuum centrifuge (Speedvac 5301). The dry residue e.g. is dissolved in 250 μL of 10% v/v Acetonitrile in water containing 0.1% formic acid. The subsequent analysis is carried out e.g. by high-pressure liquid chromatography/tandem mass spectrometry (HPLC/MS-MS) using an Agilent 1100 Series (Agilent, Palo Alto, Calif., USA) HPLC system with vacuum degasser, binary pump, and thermostated column compartment combined with a cooled autosampler system (HTS PAL, CTC Analytics, Zwingen, Switzerland). The sample (5-15 μL) is injected e.g. onto an Ultra Phenyl column (particle size 3 μm, 50×1 mm; Restek, Bellefonte, USA) with a guard column (4×2 mm) of the same material (Phenomenex, Torrance, USA). After equilibration e.g. with water and a latency period of 1 min the sample is eluted e.g. by a linear gradient of 0-100% acetonitrile in water containing 0.2% v/v formic acid over a period of 11 min at a flow rate of 60 μL/min. The column is prepared for the next sample e.g. by re-equilibrating for 3 min with 100% water to the starting conditions. The separation is performed e.g. at a column temperature of 40° C. The column effluent is introduced e.g. directly into the ion source of a triple stage quadropole mass spectrometer (Quattro Ultima™, Micromass, Manchester, UK) controlled by Masslynx™ 3.5 software (Micromass, Manchester, UK) using as ionization technique electrospray ionization positive mode (ESI+). The compound is detected by MS/MS following fragmentation of the parent ions. The limit of quantitation is determined at e.g. 0.002 nmol/L. A calibration curve is constructed with known amounts of compound including a fixed amount of internal standard in plasma which is processed as described above. The concentration of unknown samples is calculated from a plot of the peak area ratio of the selected daughter ion of the analyte to the product of its internal standard (ordinate) against the nominal concentration (abscissa). Regression analysis is performed using Quanlynx™, Masslynx™ software 3.5 (Micromass, Manchester, UK).

A dihydroimidazole of formula rac-(I) can be administered alone or in combination with one or more other therapeutic agents, possible combination therapy taking the form of fixed combinations or the administration of a compound of the invention and one or more other therapeutic agents being staggered or given independently of one another, or the combined administration of fixed combinations and one or more other therapeutic agents. A dihydroimidazole of formula rac-(I) can besides or in addition be administered especially for tumor therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Therapeutic agents for possible combination are especially one or more antiproliferative, cytostatic or cytotoxic compounds, for example a chemotherapeutic agent or several agents selected from the group which includes, but is not limited to, an inhibitor of polyamine biosynthesis, an inhibitor of a protein kinase, especially of a serine/threonine protein kinase, such as protein kinase C, or of a tyrosine protein kinase, such as the EGF receptor tyrosine kinase, the VEGF receptor tyrosine kinase, e.g. PTK787, Avastin®, or the PDGF receptor tyrosine kinase, e.g. STI571, a cytokine, a negative growth regulator, such as TGF-β or IFN-β, an aromatase inhibitor, e.g. letrozole or anastrozole, an inhibitor of the interaction of an SH2 domain with a phosphorylated protein, antiestrogens, topoisomerase I inhibitors, such as irinotecan, topoisomerase II inhibitors, microtubule active agents, e.g. paclitaxel, discodermolide or an epothilone, alkylating agents, antineoplastic antimetabolites, such as gemcitabine or capecitabine, platin compounds, such as carboplatin or cisplatin, anti-angiogenic compounds, gonadorelin agonists, anti-androgens, bisphosphonates, e.g. AREDIA® or ZOMETA®, and trastuzumab. The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

According to the present invention, a dihydroimidazole of formula rac-(I) or a tautomer thereof, or a pharmaceutically acceptable salt of such a dihydroimidazole or its tautomer, can be used in the treatment of an animal, preferably a warm-blooded animal, especially a human, that suffers from a proliferative disease. More specifically, the invention relates to the use of a dihydroimidazole of formula rac-(I) or a tautomer thereof, or a pharmaceutically acceptable salt of such a dihydroimidazole or its tautomer, in the manufacture of a medicament for the treatment of a proliferative diseases.

Furthermore, the invention provides a method for the treatment of a disease that responds to modulation of the interaction of the MDM2 protein with a p53-like peptide, which comprises administering a dihydroimidazole of formula rac-(I) or a tautomer thereof, or a pharmaceutically acceptable salt of such a dihydroimidazole or its tautomer in a quantity effective against said disease, to a warm-blooded animal requiring such treatment.

A compound of the invention may be prepared by processes that, though not applied hitherto for the new compounds of the present invention, are known per se, especially a process characterized in that for the synthesis of a compound of the formula rac-(I) wherein m is 0 and the other symbols and radicals have the meanings as defined for a compound of formula I, wherein a compound of formula rac-(II)

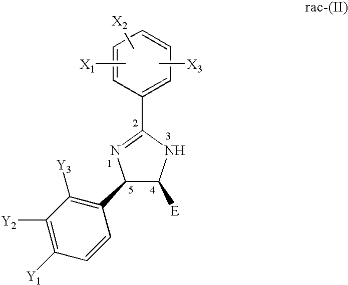

wherein the symbols and radicals have the meanings as defined for a compound of formula rac-(I), is reacted in a first step with a suitable reagent to replace the hydrogen at the ring nitrogen by a protection group PG and in a second step after deprotonation with a strong base, such as butyl lithium, with carbon dioxide to provide a carboxylic acid of formula rac-(III)

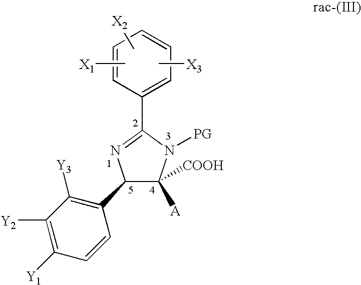

wherein the symbols and radicals have the meanings as defined for a compound of formula rac-(I). Splitting off the protection group PG under suitable reaction conditions provides the compounds of formula rac-(I);

where the above starting compounds of formula rac-(II) and rac-(III) may also be present with further functional groups in protected form if necessary and/or in the form of salts, provided a salt-forming group is present and the reaction in salt form is possible;

any additional protecting groups in a protected derivative of a compound of the formula rac-(I) are removed;

and, if so desired, an obtainable compound of formula rac-(I) is converted into another compound of formula rac-(I), a free compound of formula rac-(I) is converted into a salt, an obtainable salt of a compound of formula rac-(I) is converted into the free compound or another salt, and/or a mixture of isomeric compounds of formula rac-(I) is separated into the individual isomers.

Other dihydroimidazoles of formula rac-(I) can be prepared from the obtained dihydroimidazoles of formula rac-(I) via alkylation or acylation reaction known in the art.

Starting materials of formula rac-(II) are known from the prior art, in particular US 2004/0259867, US 2004/0259884, WO03/051360A1, WO03/051359A1 and WO2005/110996A1, or can be prepared in analogy to the methods as described therein.

The protecting groups may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of such functional groups by such protecting groups, the protecting groups themselves, and their removal reactions are described for example in standard reference books for peptide synthesis as cited hereinbefore, and in special books on protective groups such as J. F. W. McOmie, “Protective Groups in Organic Chemistry”, Plenum Press, London and New York 1973, in “Methoden der organischen Chemie” (Methods of organic chemistry), Houben-Weyl, 4th edition, Volume 15/I, Georg Thieme Verlag, Stuttgart 1974, and in T. W. Greene, “Protective Groups in Organic Synthesis”, Wiley, N.Y.

Salts can usually be converted to free compounds, e.g. by treating with suitable basic agents, for example with alkali metal carbonates, alkali metal hydrogencarbonates, or alkali metal hydroxides, typically potassium carbonate or sodium hydroxide.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

All process steps described here can be carried out under known reaction conditions, preferably under those specifically mentioned, in the absence of or usually in the presence of solvents or diluents, preferably such as are inert to the reagents used and able to dissolve these, in the absence or presence of catalysts, condensing agents or neutralising agents, for example ion exchangers, typically cation exchangers, for example in the H⁺ form, depending on the type of reaction and/or reactants at reduced, normal, or elevated temperature, for example in the range from −100° C. to about 190° C., preferably from about −80° C. to about 150° C., for example at −80 to −60° C., at room temperature, at −20 to 40° C. or at the boiling point of the solvent used, under atmospheric pressure or in a closed vessel, where appropriate under pressure, and/or in an inert atmosphere, for example under argon or nitrogen.

Salts may be present in all starting compounds and transients, if these contain salt-forming groups. Salts may also be present during the reaction of such compounds, provided the reaction is not thereby disturbed.

At all reaction stages, isomeric mixtures that occur can be separated into their individual isomers, e.g. diastereomers or enantiomers, or into any mixtures of isomers, e.g. racemates or diastereomeric mixtures.

The invention relates also to those forms of the process in which one starts from a compound obtainable at any stage as a transient and carries out the missing steps, or breaks off the process at any stage, or forms a starting material under the reaction conditions, or uses said starting material in the form of a reactive derivative or salt, or produces a compound obtainable by means of the process according to the invention and processes the said compound in situ. In the preferred embodiment, one starts from those starting materials which lead to the compounds described hereinabove as preferred, particularly as especially preferred, primarily preferred, and/or preferred above all.

In the preferred embodiment, a dihydroimidazole of formula rac-(I) is prepared according to or in analogy to the processes and process steps defined in the Examples.

The present invention relates also to pharmaceutical compositions that comprise a dihydroimidazole of formula rac-(I) as active ingredient and that can be used especially in the treatment of the diseases mentioned at the beginning. Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions comprise the active ingredient alone or, preferably, together with a pharmaceutically acceptable carrier. The dosage of the active ingredient depends upon the disease to be treated and upon the species, its age, weight, and individual condition, the individual pharmacokinetic data, and the mode of administration.

The present invention relates especially to pharmaceutical compositions that comprise a dihydroimidazole of formula rac-(I), a tautomer, or a pharmaceutically acceptable salt, and at least one pharmaceutically acceptable carrier.

The invention relates also to pharmaceutical compositions for use in a method for the prophylactic or especially therapeutic management of the human or animal body, to a process for the preparation thereof (especially in the form of compositions for the treatment of tumors) and to a method of treating tumor diseases, especially those mentioned hereinabove.

The invention relates also to processes and to the use of dihydroimidazole of formula rac-(I) for the preparation of pharmaceutical preparations which comprise dihydroimidazoles of formula rac-(I) as active component (active ingredient).

The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories, or capsules. Further dosage forms are, for example, ointments, creams, pastes, foams, tinctures, lip-sticks, drops, sprays, dispersions, etc. Examples are capsules containing from about 0.05 g to about 1.0 g active ingredient.

The pharmaceutical compositions of the present invention are prepared in a manner known per se, for example by means of conventional mixing, granulating, coating, dissolving or lyophilizing processes.

Pharmaceutical compositions for oral administration can be obtained, for example, by combining the active ingredient with one or more solid carriers, if desired granulating a resulting mixture, and processing the mixture or granules, if desired or necessary, by the inclusion of additional excipients, to form tablets or tablet cores.

Solutions such as are used, for example, for parenteral administration can also be employed as infusion solutions.

The invention relates likewise to a process or a method for the treatment of one of the pathological conditions mentioned hereinabove, especially a corresponding neoplastic disease. The dihydroimidazoles of formula rac-(I) or pharmaceutically acceptable salts thereof can be administered as such or especially in the form of pharmaceutical compositions, prophylactically or therapeutically, preferably in an amount effective against the said diseases, to a warm-blooded animal, for example a human, requiring such treatment. In the case of an individual having a bodyweight of about 70 kg the daily dose administered is from approximately 0.05 g to approximately 5 g, preferably from approximately 0.25 g to approximately 1.5 g, of a compound of the present invention.

The following Examples serve to illustrate the invention without limiting the invention in its scope.

### Abbreviations:

AcOH acetic acid
BEMP 2-tert-butylimino-2-diethylamino-1,3-dimethyl-perhydro-1,3,2-diazaphosphorine
BOC benzyloxy carbonyl
DMSO dimethylsulfoxide
EtOAc ethyl acetate
EtOH ethanol
HPLC high pressure liquid chromatography
LC-MS liquid chromatography-mass spectrometry
m.p. melting point-
MPLC medium pressure liquid chromatography
MS mass spectra
MW molecular weight
PTFA polytetrafluoroethylene
TFA trifluoro acetic acid
TLC thin layer chromatogram

The following Examples serve to illustrate the invention without limiting the invention in its scope. Temperatures are measured in degrees celsius (° C.). Unless otherwise indicated, the reactions take place at room temperature.

### Examples

The following analytical systems were used:

Preparative MPLC (System 1): Labomatic MPLC system with linear gradient: hexane/tert-butyl methyl ether 90:10 to 50:50 in 50 min followed by 40 min elution with hexane/tert-butyl methyl ether 50:50, column: 200 g silicagel normal phase.

Analytic LC-MS system (System 2): Waters 2795 HPLC system with Micromass ZQ MS detection—Gradient water with 0.05% TFA/acetonitrile with 0.05% TFA, 95:5 to 5:95 in 8 min, linear gradient, flow: 1.4 mL/min, column: Macherey-Nagel 70/4.6 Nucleosil 100-3 C18, Pre-column: CC 8/3 Nucleosil 100-3 C18.

Preparative LC-MS (System 3): Waters 2525 HPLC system with Micromass ZQ MS detection. One minute elution with 5% aqueous acetonitrile containing 0.1% of TFA followed by linear gradient of 7 min from 5% aqueous acetonitrile to 95% aqueous acetonitrile, both containing 0.1% TFA, using a flow rate of 30 mL/min on a Waters Sunfire™ prep C-18 column 19×100 mm, 5 um. The desired products are collected in one fraction, based on mass detection.

Analytic LC-MS (System 4): Waters 2795 HPLC system with Micromass ZQ MS detection—Gradient water with 0.05% TFA/acetonitrile with 0.05% TFA, 95:5 to 5:95 in 4 min, linear gradient, flow: 1.8 mL/min, column: Macherey-Nagel 70/4.6 Nucleosil 100-3 C18, Pre-column: CC 8/3 Nucleosil 100-3 C18.

Preparative LC-MS system (System 5): Waters 2525 HPLC system with Micromass ZQ MS detection. Two minutes elution with 20% aqueous acetonitrile containing 0.1% TFA followed by linear gradient of 8 min from 20% aqueous acetonitrile to 75% aqueous acetonitrile, both containing 0.1% TFA, using a flow rate of 30 ml/min on a Waters Sunfire™ prep C-18 column 19×100 mm, 5 um. The desired products are collected based on mixed fraction trigger mass and UV (220 nm).

Preparative LC-MS system (System 6): Waters 2525 HPLC system with Micromass ZQ MS detection. One minute elution with 5% aqueous acetonitrile containing 0.1% of TFA followed by linear gradient of 7 min from 20% aqueous acetonitrile to 80% aqueous acetonitrile, both containing 0.1% TFA, using a flow rate of 30 mL/min on a Waters Sunfire™ prep C-18 column 19×100 mm, 5 um. The desired products are collected in one fraction, based on mass detection.

Analytical LC-MS system (System 7): Waters 2795 HPLC system with Micromass ZQ MS detection—Gradient water with 0.05% TFA/acetonitrile with 0.05% TFA, 95:5 to 5:95 in 8 min, linear gradient, flow: 1.4 mL/min followed by 2 min elution with 95% water with 0.05% TFA, column: Macherey-Nagel 70/4.6 Nucleosil 100-3 C18, Pre-column: CC 8/3 Nucleosil 100-3 C18.

Preparative LC-MS system (System 8): Waters 2525 HPLC system with Micromass ZQ MS detection. Aqueous acetonitrile of the following composition containing 0.1% of trifluoroacetic acid is used as a mobile phase at a flow rate of 30 ml/min on a Waters Sunfire™ C-18 column 19×100 mm, 5 μm: linear gradient of 1.5 minutes from 5% aqueous acetonitrile to 50% aqueous acetonitrile, followed by a linear gradient of 7.5 minutes from 50% aqueous acetonitrile to 95% aqueous acetonitrile, followed by a linear gradient of 1.0 minute from 95% aqueous acetonitrile to 100% acetonitrile. The collection of products is triggered by the MS signal. Analytical LC-MS system (System 9): Agilent 1100 LC HPLC system with Micromass ZMD MS detection. A binary gradient composed of A (water containing 5% acetonitrile and 0.2% formic acid) and B (acetonitrile containing 0.2% formic acid) is used as a mobile phase at a flow rate of 0.7 ml/min on a Waters×Terra™ C-18 column 3×30 mm, 2.5 μm: isocratic elution during 0.5 minutes of 95% of A followed by a linear gradient of 3.0 minutes from 95% to 5% of A followed by isocratic elution during 1.0 minute of 5% of A.

### Example 1

### Trans-4,5-Bis-(4-chloro-phenyl)-2-(2-isopropoxy-4-methoxy-phenyl)-4,5-dihydro-3H-imidazole-4-carboxylic acid ethyl ester trifluoroacetic acid salt

In a 500 mL round bottom flask, trans-4,5-bis-(4-chloro-phenyl)-2-(2-isopropoxy-4-methoxy-phenyl)-4,5-dihydro-imidazole-1,5-dicarboxylic acid 1-tert-butyl ester 5-ethyl ester (Step 1.9, 14 g, 22.3 mmol), is added to an ice cold solution of 10% v/v trifluoroacetic acid in dichloromethane (100 mL). The reaction mixture is stirred at 0° C. for 0.5 h and then at room temperature for 10 h. After concentration in vacuo, the title compound is obtained as a white solid, ¹HNMR (400 Hz, DMSO-d6) δ 1.28 (3H, t), 1.37 (6H, d), 3.91 (3H, s), 4.36-4.41 (2H, m), 4.93 (1H, t), 6.16 (1H, s), 6.84 (2H, t), 7.05-7.12 (4H, m), 7.28 (4H, t), 7.86 (1H, d), 10.6 (1H, s), 11.29 (1H, s).

### Step 1.1: 2-Hydroxy-4-methoxy-benzamide

In a 500 mL round bottom flask, 4-methoxy salicylic acid (20 g, 118.9 mmol), oxalyl chloride (20.5 mL, 237.88 mmol) and dichloromethane (100 mL) are stirred at 0° C. for 0.5 h, then warmed at 40-50° C. for 2 h. Solvent and excess oxalyl chloride are evaporated under reduced pressure. The residue is again diluted with dichloromethane (200 mL) and ammonia gas is purged into this solution for 1 h, then it is stirred for 48 h at room temperature. Solvent removed and crude mass are re-dissolved in hot ethyl acetate and washed with saturated aqueous sodium carbonate solution. Ethyl acetate layer is dried over anhydrous magnesium sulfate and evaporated under reduced pressure to get the title compound as a pale yellow solid, ¹HNMR (400 Hz, DMSO-d₆) δ 3.81 (3H, s), 6.41 (2H, s), 7.73 (2H, t), 8.21 (1H, s), 13.43 (1H, s).

### Step 1.2: 2-Hydroxy-4-methoxy-benzonitrile

In a 1L round bottom flask, 2-hydroxy-4-methoxy-benzamide (Step 1.1, 19.5 g, 116.76 mmol), triethylamine (33 mL, 234.12 mmol), trifluoromethane sulfonic anhydride (23.3 mL, 140.48 mmol) and dry dichloromethane (500 mL) are mixed and the reaction mixture is stirred for 0.5 h in ice-cold condition and then at room temperature for 12 h. The reaction mixture is washed with distilled water (2×100 mL) and then the organic layer is dried over anhydrous magnesium sulfate, evaporated under reduced pressure and crude material is subjected to column chromatography, (100-200 mesh silica gel, 15% ethyl acetate in hexane) to obtain the title compound as a pale yellow solid, ¹HNMR (400 Hz, CDCl₃) δ 3.90 (3H, s), 6.97 (2H, t), 7.65 (1H, d).

### Step 1.3: 2-Isopropoxy-4-methoxy-benzonitrile

In a 250 mL round bottom flask, 2-hydroxy-4-methoxy-benzonitrile (Step 1.2, 9.11 g, 61.07 mmol), potassium carbonate (17 g, 122.3 mmol), 2-iodopropane (12 mL, 122.28 mmol) and N,N-dimethylformamide (70 mL) are stirred for 12 h at 70° C. The reaction mixture is cooled to room temperature and filtered through celite. The filtrate is diluted with water (500 mL) and extracted with 50% ethyl acetate in hexane; the organic layer is washed with water (2×200 mL), dried over anhydrous magnesium sulfate and then evaporated under reduced pressure. Finally, pure title compound is obtained by column chromatography (100-200 mesh silica gel, 10% ethyl acetate in hexane) as pale yellow solid, ¹HNMR (400 Hz, CDCl₃) δ 1.37 (6H, d), 3.82 (3H, s), 4.55-4.62 (1H, m), 6.43-6.49 (2H, m), 7.45 (1H, d).

### Step 1.4: 4-Chloro-N-[(S)-2-[(4-chloro-benzylidene)-amino]-1,2-bis-(4-chloro-phenyl)-ethyl]-benzamide

In a 1L round bottom flask, 4-chlorobenzaldehyde (100 g, 711.4 mmol)) and ammonium acetate (200 g) are heated at 200° C. for 5 h. The reaction mixture is cooled and diluted with water, obtained solid is filtered off through a sintered funnel and the solid is dried in air for several hours. It is washed with 10% ethyl acetate in hexane to remove the unreacted 4-chlorobenzaldehyde, finally the desired title compound is obtained as a crystalline white solid.

### Step 1.5: (1S,2R)-1,2-Bis-(4-chloro-phenyl)-ethane-1,2-diamine

In a 1L round bottom flask, 4-chloro-N-[(S)-2-[(4-chloro-benzylidene)-amino]-1,2-bis-(4-chloro-phenyl)-ethyl]-benzamide (Step 1.4, 80 g, 156 mmol) and 70% sulfuric acid (350 mL) are refluxed at 160° C. for 4 h. The reaction mixture is cooled and poured in crushed ice (500 g). Aqueous layer is washed with ethyl acetate to remove impurities. The pH of aqueous layer is adjusted with aqueous 4M sodium hydroxide solution and extracted with ethyl acetate. Ethyl acetate layer is dried over anhydrous magnesium sulfate, and evaporated under reduced pressure to obtain the title compound as a white crystalline solid.

### Step 1.6: 2-Isopropoxy-4-methoxy-benzimidic acid ethyl ester hydrochloride

In a 250 mL round bottom flask, 2-isopropoxy-4-methoxy-benzonitrile (Step 1.3, 16 g, 83.66 mmol) and absolute ethanol (70 mL) are taken, dry hydrogen chloride gas is passed into this solution for 1 h and stirred for 6 h at room temperature. The reaction mass is concentrated under reduced pressure to obtain a viscous liquid, which is finally crystallized from methyl t-butyl ether. The title compound is obtained as a white crystalline solid, ¹HNMR (400 Hz, DMSO-d₆) δ 1.35 (6H, d), 1.43 (3H, t), 3.88 (3H, s), 4.55 (2H, q), 4.88-4.94 (1H, m), 6.72-6.80 (2H, m), 7.75 (1H, d), 10.5 (1H, br s), 11.1 (1H, br, s).

### Step 1.7: trans-4,5-Bis-(4-chloro-phenyl)-2-(2-isopropoxy-4-methoxy-phenyl)-4,5-dihydro-1H-imidazole

In a 250 mL round bottom flask, 2-isopropoxy-4-methoxy-benzimidic acid ethyl ester hydrochloride (Step 1.6, 14 g, 51.13 mmol), (1S,2R)-1,2-bis-(4-chloro-phenyl)-ethane-1,2-diamine (Step 1.5, 14.4 g, 51.23 mol) and absolute ethanol (170 mL) are refluxed at 90° C. for 12 h. The reaction mixture is cooled and evaporated under reduced pressure. The desired compound is obtained by column chromatography (100-200 mesh silica gel, 100% ethyl acetate) as a pale yellow solid, ¹HNMR (400 Hz, CDCl₃) δ 1.37 (6H, d), 3.86 (3H, s), 4.70-4.73 (1H, m), 5.40 (2H, s), 6.53 (1H, s), 6.63 (1H, dd), 6.89 (4H, d), 7.03 (4H, d), 8.39 (1H, d).

### Step 1.8: trans-4,5-Bis-(4-chloro-phenyl)-2-(2-isopropoxy-4-methoxy-phenyl)-4,5-dihydro-imidazole-1-carboxylic acid tert-butyl ester

In a 500 mL round bottom flask, 4,5-bis-(4-chlorophenyl)-2-(2-isopropoxy-4-methoxy-phenyl)-4,5-dihydro-1H-imidazole (Step 1.7, 15.5 g, 34 mmol), dimethyl amino pyridine (6.2 g, 51 mmol), di-tert-butyldicarbonate (15.6 mL, 68 mmol) and tetrahydrofuran (150 mL) are stirred at room temperature for 12 h. The reaction mixture is evaporated under reduced pressure and the title compound is obtained by column chromatography (100-200 mesh silica gel, 10% ethyl acetate) as a pale yellow solid, ¹HNMR (400 Hz, CDCl₃) δ 1.16 (9H, s), 1.32 (3H, d), 1.42 (3H, d), 3.82 (3H, s), 4.66 (1H, t), 5.50 (1H, d), 5.60 (1H, d), 6.48 (2H, d), 6.94-7.08 (8H, m), 7.43 (1H, d).

### Step 1.9: trans-4,5-bis-(4-chloro-phenyl)-2-(2-isopropoxy-4-methoxy-phenyl)-4,5-dihydro-imidazole-1,5-dicarboxylic acid 1-tert-butyl ester 5-ethyl ester

To a 500 mL two neck round bottom flask, a solution of tert-butyllithium (1.7M in tetrahydrofuran) (25.42 mL, 43.21 mmol) is added to cold (−78° C.), stirred solution of trans-4,5-bis-(4-chloro-phenyl)-2-(2-isopropoxy-4-methoxy-phenyl)-4,5-dihydro-imidazole-1-carboxylic acid tert-butyl ester (Step 1.8, 16 g, 28.8 mmol) in dry tetrahydrofuran (60 mL) under an atmosphere of argon. The mixture is warmed to −40° C. After 0.5 h, the reaction mixture is again cooled to −78° C. and ethyl chloroformate (3.75 g, 34.56 mmol)) dissolved in dry tetrahydrofuran (30 mL) is added. Then the reaction mixture is stirred at room temperature for 12 h. The reaction is quenched with saturated aqueous ammonium chloride solution. After concentration, the residue is extracted with ethyl acetate. The crude product is purified by column chromatography (neutral alumina, 3% ethyl acetate in hexane), ¹HNMR (400 Hz, CDCl₃) δ 1.15 (9H, s), 1.20-1.25 (6H, m), 1.36 (3H, d), 3.82 (3H, s), 4.23-4.28 (2H, q), 4.62 (1H, t), 6.12 (1H, s), 6.46-6.50 (2H, m), 6.99-7.01 (4H, m), 7.06-7.10 (4H, m), 7.40 (2H, d).

### Example 2

### 1-(4-Acetyl-piperazine-1-carbonyl)-trans-4,5-bis-(4-chloro-phenyl)-2-(2-isopropoxy-4-methoxy-phenyl)-4,5-dihydro-1H-imidazole-4-carboxylic acid ethyl ester

A solution of 1-chlorocarbonyl-trans-4,5-bis-(4-chloro-phenyl)-2-(2-isopropoxy-4-methoxy-phenyl)-4,5-dihydro-1H-imidazole-4-carboxylic acid ethyl ester (30 mg, 0.051 mmol) in 1,2-dichloroethane (1 mL) is added to a stirred mixture of 1-acetylpiperazine (10.2 mg, 0.08 mmol) and triethylamine (28.4 μL, 0.203 mmol) cooled to 5° C. The reaction mixture is stirred at 5° C. for 30 min. The solution is evaporated under reduced pressure. The crude product is purified using a preparative LC-MS system (System 3) to give the desired compound, LC-MS (System 4) ([M+H]⁺=681.25, retention time=3.22 min).

### Step 2.1: trans-4,5-Bis-(4-chloro-phenyl)-2-(2-isopropoxy-4-methoxy-phenyl)-4,5-dihydro-1H-imidazole-4-carboxylic acid ethyl ester

Trans-4,5-Bis-(4-chloro-phenyl)-2-(2-isopropoxy-4-methoxy-phenyl)-4,5-dihydro-1H-imidazole-4-carboxylic acid ethyl ester trifluoroacetate salt (Example 1, 1.746 g, 2.722 mmol) is dissolved in ethyl acetate (100 mL) and extracted four times (4×50 mL) with saturated aqueous potassium carbonate solution. The organic layer is dried over sodium sulfate, filtered and concentrated under vacuum to obtain trans-4,5-Bis-(4-chloro-phenyl)-2-(2-isopropoxy-4-methoxy-phenyl)4,5-dihydro-1H-imidazole-4-carboxylic acid ethyl ester free base as a pale yellow powder. The isolated product is identified by LC-MS (system 4) ([M+H]⁺=527.08, retention time=3.28 min).

### Step 2.2: 1-Chlorocarbonyl-trans-4,5-bis-(4-chloro-phenyl)-2-(2-isopropoxy-4-methoxy-phenyl)-4,5-dihydro-1H-imidazole-4-carboxylic acid ethyl ester

To a solution of trans-4,5-bis-(4-chloro-phenyl)-2-(2-isopropoxy-4-methoxy-phenyl)-4,5-dihydro-1H-imidazole-4-carboxylic acid ethyl ester (Step 2.1, 2.5 g, 4.74 mmol) in dichloromethane (200 mL) cooled to 0° C. is sequentially added triethylamine (1.33 mL, 9.54 mmol) and phosgene (7.0 mL, 14.2 mmol, 20% in toluene). The reaction mixture is stirred at 0° C. under argon for 30 min. The solvent and excess reagents are evaporated under reduced pressure and the residue is purified on a MPLC preparative system (System 1) to give the desired compound as a white foam, LC-MS (System 2) ([M+H]⁺=589.01, retention time=7.47 min).

### Example 3

### Ureas Derivatives

A solution of 1-chlorocarbonyl-trans-4,5-bis-(4-chloro-phenyl)-2-(2-isopropoxy-4-methoxy-phenyl)-4,5-dihydro-1H-imidazole-4-carboxylic acid ethyl ester (Step 2.2, 15 mg, 0.025 mmol) in 1,2-dichloroethane (0.5 mL) is added to a solution of different amines (1.5 eq.) and triethylamine (4 eq.) in dichloromethane (0.5 mL) cooled at 5° C. The reaction mixture is stirred at 5° C. for 30 min. The solutions are evaporated with a needle air stream evaporator. The residues are purified using a preparative LC-MS system (System 5) and controlled by LC-MS (SYSTEM 4)

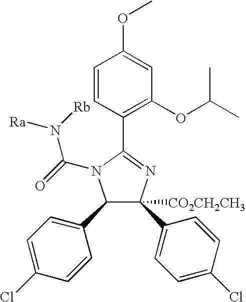

| | | | MW of the | Retention |
|---|---|---|---|---|
| | | | final | time (min) |
| | | | compounds | of LC-MS |
| | | | as free | (SYSTEM |
| Ex. | HNRaRb | Supplier | bases | 4) |
| 3.1 | 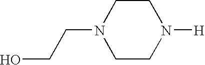 | FLUKA | 683.64 | 2.92 |
| 3.2 | 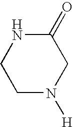 | ALDRICH | 653.57 | 3.05 |
| 3.3 | 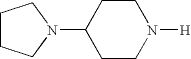 | ALDRICH | 707.70 | 2.91 |
| 3.4 | 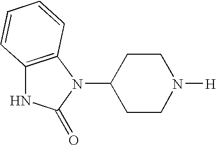 | ALDRICH | 770.72 | 3.29 |
| 3.5 | 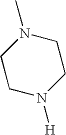 | ALDRICH | 653.61 | 2.98 |
| 3.6 | 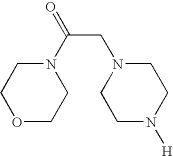 | OAKWOOD | 766.73 | 3.00 |
| 3.7 | 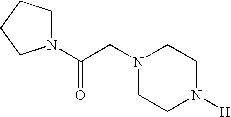 | OAKWOOD | 750.73 | 3.07 |
| 3.8 | 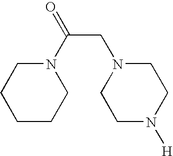 | OAKWOOD | 764.76 | 3.15 |
| 3.9 | 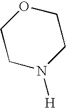 | ALDRICH | 640.57 | 3.28 |
| 3.10 | 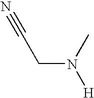 | ALDRICH | 623.54 | 3.33 |
| 3.11 | 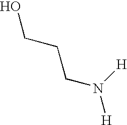 | ALDRICH | 628.56 | 3.01 |
| 3.12 | 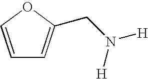 | ALDRICH | 650.56 | 3.35 |
| 3.13 | 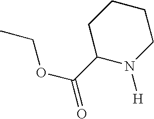 | ALDRICH | 710.66 | 3.54 |
| 3.14 | 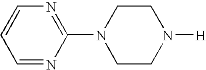 | ALDRICH | 717.66 | 3.41 |
| 3.15 | 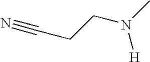 | ALDRICH | 637.57 | 3.23 |
| 3.16 | 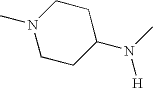 | JW- PHARMLAB | 681.67 | 2.80 |
| 3.17 | 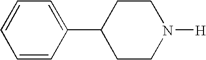 | ALDRICH | 714.70 | 3.63 |
| 3.18 | 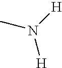 | FLUKA | 584.50 | 3.18 |
| 3.19 | 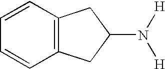 | ALDRICH | 686.64 | 3.55 |
| 3.20 | 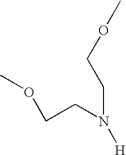 | FLUKA | 686.64 | 3.41 |
| 3.21 | 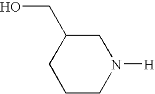 | ALDRICH | 668.62 | 3.19 |
| 3.22 | 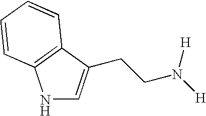 | ALDRICH | 713.67 | 3.43 |
| 3.23 | 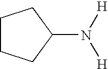 | ALDRICH | 638.60 | 3.46 |
| 3.24 | 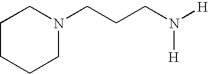 | ABCR | 695.69 | 2.88 |
| 3.25 | 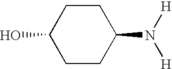 | ALDRICH | 668.62 | 3.05 |
| 3.26 | 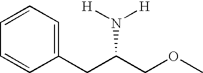 | ALDRICH | 718.68 | 3.59 |
| 3.27 | 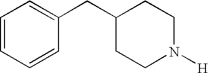 | ALDRICH | 728.72 | 3.70 |
| 3.28 | 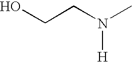 | ALDRICH | 628.56 | 3.07 |
| 3.29 | 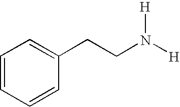 | ALDRICH | 674.63 | 3.51 |
| 3.30 | 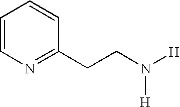 | ASDI | 689.65 | 2.88 |
| 3.31 | 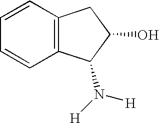 | ALDRICH | 702.64 | 3.38 |
| 3.32 | 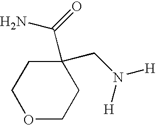 | ASDI | 711.65 | 2.97 |
| 3.33 | 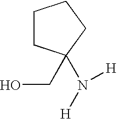 | ALDRICH | 668.62 | 3.30 |
| 3.34 | 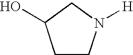 | ALDRICH | 640.57 | 3.08 |
| 3.35 | 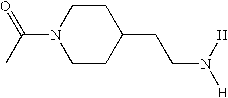 | ASDI | 723.70 | 3.16 |
| 3.36 | 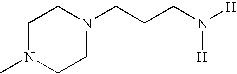 | LANCASTER | 710.71 | 2.63 |
| 3.37 | 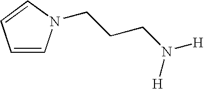 | RARECHEM | 677.63 | 3.38 |
| 3.38 | 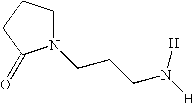 | ALDRICH | 695.65 | 3.15 |
| 3.39 | 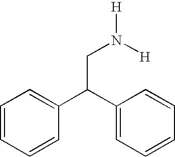 | ALDRICH | 750.73 | 3.68 |
| 3.40 | 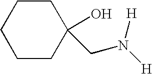 | ALDRICH | 682.65 | 3.36 |
| 3.41 | 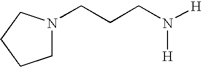 | ACROS | 681.67 | 2.83 |
| 3.42 | 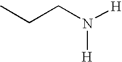 | ALDRICH | 612.56 | 3.34 |
| 3.43 | 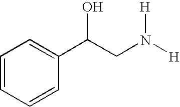 | ALDRICH | 690.63 | 3.30 |
| 3.44 | 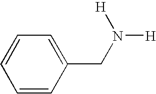 | ACROS | 660.60 | 3.43 |
| 3.45 | 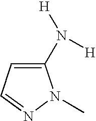 | APOLLO | 650.57 | 3.34 |
| 3.46 | 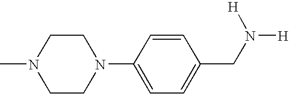 | MAYBRIDGE | 758.75 | 2.83 |
| 3.47 | 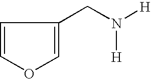 | MAYBRIDGE | 650.56 | 3.31 |
| 3.48 | 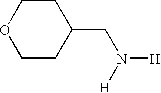 | MAYBRIDGE | 668.62 | 3.21 |
| 3.49 | 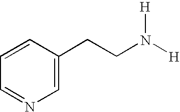 | ALFA AESAR | 675.62 | 2.79 |
| 3.50 | 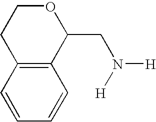 | ASDI | 716.67 | 3.56 |
| 3.51 | 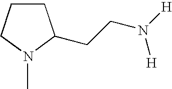 | ALDRICH | 681.67 | 2.80 |
| 3.52 | 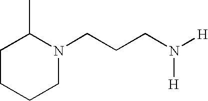 | ALDRICH | 709.72 | 2.93 |
| 3.53 | 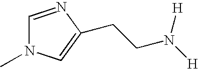 | ALDRICH | 678.62 | 2.78 |
| 3.54 | 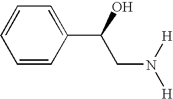 | ALDRICH | 690.63 | 3.31 |
| 3.55 | 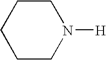 | ALDRICH | 638.60 | 3.47 |
| 3.56 | 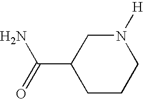 | ALDRICH | 681.62 | 3.06 |
| 3.57 | 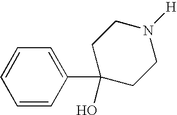 | ALDRICH | 730.70 | 3.36 |
| 3.58 | 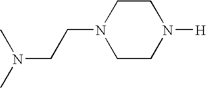 | FLUKA | 710.71 | 2.83 |
| 3.59 | 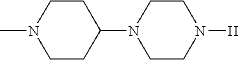 | FLUKA | 736.75 | 2.76 |
| 3.60 | 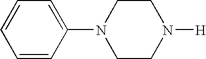 | ALDRICH | 715.68 | 3.57 |
| 3.61 | 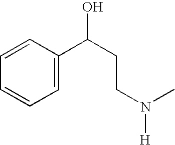 | ALDRICH | 718.68 | 3.35 |
| 3.62 | 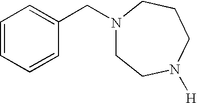 | ASDI | 743.74 | 3.09 |
| 3.63 | 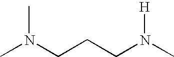 | ALDRICH | 669.65 | 2.78 |
| 3.64 | 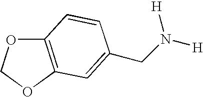 | ALDRICH | 704.61 | 3.37 |
| 3.65 | 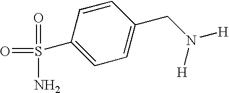 | TCI EUROPE | 739.68 | 3.04 |
| 3.66 | 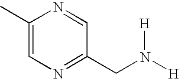 | TCI EUROPE | 676.61 | 3.20 |

### Example 4

### Trans-4,5-Bis-(4-chlorophenyl)-2-(2-isopropoxy-4-methoxyphenyl)-4,5-dihydro-3H-imidazole-4-carboxylic acid (pyridin-4-ylmethyl)-amide trifluoroacetate salt

To a stirred solution of trans-4,5-bis-(4-chlorophenyl)-2-(2-isopropoxy-4-methoxyphenyl)-4,5-dihydro-3H-imidazole-4-carboxylic acid (Step 4.1; 50 mg, 0.1 mmol) in 250 μL of N,N-dimethylformamide are added O-benzotriazol-1-yl-N,N,N′,N′-tetramethyluronium hexafluorophosphate (76 mg, 0.2 mmol) and N,N-diisopropylethylamine (35 μL, 0.2 mmol). The reaction is stirred for one hour at room temperature. A solution of 4-aminomethylpiridine (21 μL, 0.2 mmol) with N,N-diisopropylethylamine (35 μL, 0.2 mmol) in 250 μL of N,N-dimethylformamide is added. The reaction is stirred overnight at room temperature. The crude is directly purified using preparative LC-MS system (System 3) to give the desired compound, LC-MS (SYSTEM 2) ([M+H]⁺=598.09, retention time=2.63 min).

### Step 4.1: trans-4,5-Bis-(4-chlorophenyl)-2-(2-isopropoxy-4-methoxyphenyl)-4,5-dihydro-3H-imidazole-4-carboxylic acid

To a solution of trans-4,5-bis-(4-chlorophenyl)-2-(2-isopropoxy-4-methoxyphenyl)-4,5-dihydro-3H-imidazole-4-carboxylic acid ethyl ester trifluoroacetate salt (Example 1, 1 g, 1.56 mmol) in water (3 mL) and ethanol (3 mL) is added 1N potassium hydroxide solution (7.8 mL, 7.8 mmol). The reaction is carried out under sealed-vessel microwave heating at 120° C. for 10 minutes using a Biotage Initiator™ (pre-stirring: 15 s, absorption level: high). The reaction mixture is diluted in ethyl acetate (25 mL) and then neutralized with solid citric acid up to pH=7. The aqueous layer is extracted with ethyl acetate (3×25 mL). The combined organic layers are washed with brine (30 mL), dried over anhydrous sodium sulfate, filtered and concentrated under vacuum to give the desired compound as a pale white solid. The isolated product is identified by LC-MS (System 4) ([M+H]⁺=499.01, retention time=3.03 min).

### Example 5

### Amide Derivatives

To a stirred solution of trans-4,5-bis-(4-chlorophenyl)-2-(2-isopropoxy-4-methoxyphenyl)-4,5-dihydro-3H-imidazole-4-carboxylic acid (Example 1, 1.68 g, 3.36 mmol) in 22 mL of N,N-dimethylformamide are added O-benzotriazol-1-yl-N,N,N′,N′-tetramethyluronium hexafluorophosphate (2.55 g, 6.73 mmol) and N,N-diisopropylethylamine (2.35 mL, 13.5 mmol). The reaction is stirred one hour at room temperature. This solution is then distributed over each tube of a 96 well rack (250 μL, 0.04 mmol). To each tube N,N-diisopropylethylamine (14 μL, 0.08 mmol) is added, followed by one of the 85 amines (0.08 mmol, 2 eq). The rack is shaken overnight at room temperature. The crude products are purified using a preparative LC-MS system (System 3).

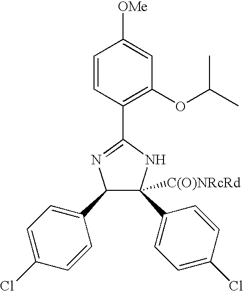

| | | | MW of the | Retention |
|---|---|---|---|---|
| | | | final | time (min) |
| | | | compounds | of LC-MS |
| | Amines | | as free | (SYSTEM |
| Ex. | HNRcRd | Supplier | bases | 4) |
| 5.1 | 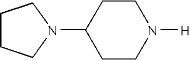 | ALDRICH | 635.64 | 2.59 |
| 5.2 | 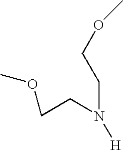 | FLUKA | 614.57 | 3.28 |
| 5.3 | 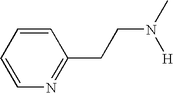 | ASDI | 617.58 | 2.7 |
| 5.4 | 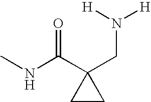 | ASDI | 609.56 | 2.98 |
| 5.5 | 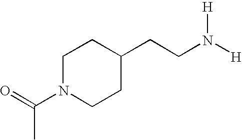 | ASDI | 651.64 | 3.03 |
| 5.6 | 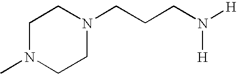 | LANCASTER | 638.64 | 2.48 |
| 5.7 | 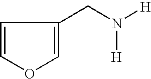 | MAYBRIDGE | 578.50 | 3.17 |
| 5.8 | 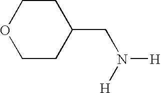 | MAYBRIDGE | 596.56 | 3.08 |
| 5.9 | 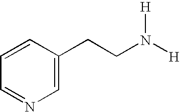 | ALFA AESAR | 603.55 | 2.63 |
| 5.10 | 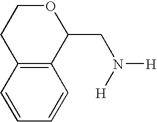 | ASDI | 644.60 | 3.36 |
| 5.11 | 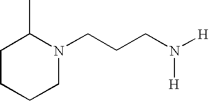 | ALDRICH | 637.66 | 2.79 |
| 5.12 | 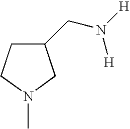 | ALDRICH | 595.57 | 2.55 |
| 5.13 | 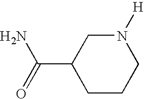 | ALDRICH | 609.56 | 2.89 |
| 5.14 | 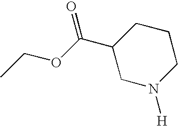 | ALDRICH | 638.60 | 3.35 |
| 5.15 | 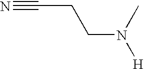 | ALDRICH | 565.50 | 3.02 |
| 5.16 | 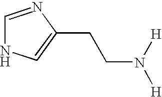 | ASDI | 592.53 | 2.58 |
| 5.17 | 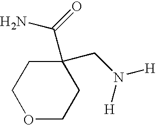 | ASDI | 639.58 | 2.89 |
| 5.18 | 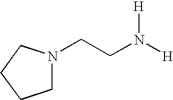 | ACROS | 595.57 | 2.68 |
| 5.19 | 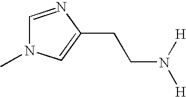 | ALDRICH | 606.56 | 2.65 |
| 5.20 | 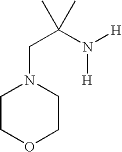 | ACROS | 639.63 | 2.78 |
| 5.21 | 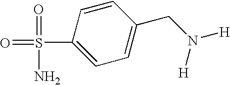 | TCI-EUROPE | 667.62 | 2.95 |
| 5.22 | 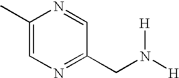 | TCI-EUROPE | 604.54 | 3.03 |
| 5.23 | 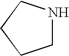 | ALDRICH | 552.51 | 3.18 |
| 5.24 | 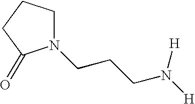 | ALDRICH | 623.59 | 3.07 |
| 5.25 | 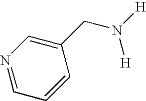 | ALDRICH | 589.53 | 2.66 |
| 5.26 | 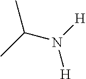 | ALDRICH | 540.49 | 3.16 |
| 5.27 | 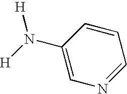 | ALDRICH | 575.50 | 2.83 |
| 5.28 | 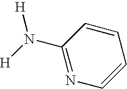 | ALDRICH | 575.50 | 3.2 |
| 5.29 | 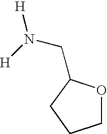 | ALDRICH | 582.53 | 3.14 |
| 5.30 | 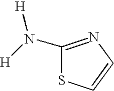 | ALDRICH | 581.53 | 3.16 |
| 5.31 | 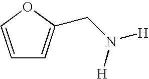 | ALDRICH | 578.50 | 3.19 |
| 5.32 | 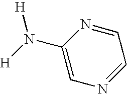 | ALDRICH | 576.49 | 3.11 |
| 5.33 | 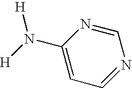 | ACROS | 576.49 | 3.03 |
| 5.34 | 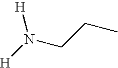 | ALDRICH | 540.49 | 3.19 |
| 5.35 | 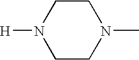 | ALDRICH | 581.55 | 2.55 |
| 5.36 | 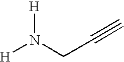 | ACROS | 536.46 | 3.23 |
| 5.37 | 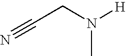 | ALDRICH | 551.48 | 3.04 |
| 5.38 | 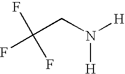 | ALDRICH | 580.44 | 3.18 |
| 5.39 | 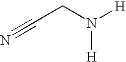 | ALFA AESAR | 537.45 | 3.01 |
| 5.40 | 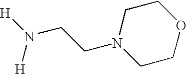 | ACROS | 611.57 | 2.67 |
| 5.41 | 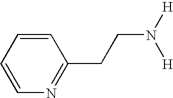 | ALDRICH | 603.55 | 2.69 |
| 5.42 | 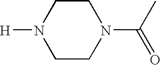 | ABCR | 609.56 | 2.91 |
| 5.43 | 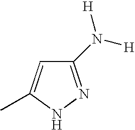 | ALDRICH | 578.50 | 3.2 |
| 5.44 | 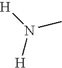 | ALDRICH | 512.44 | 3.05 |
| 5.45 | 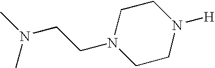 | ABCR | 638.64 | 2.55 |
| 5.46 | 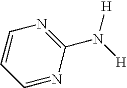 | ALDRICH | 576.49 | 3.01 |
| 5.47 | 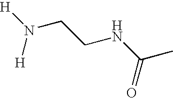 | ALDRICH | 583.52 | 2.91 |
| 5.48 | 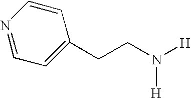 | ABCR | 603.55 | 2.59 |
| 5.49 | 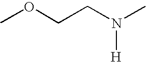 | ABCR | 570.52 | 3.16 |
| 5.50 | 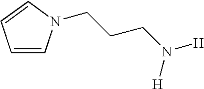 | RARE CHEM | 605.57 | 3.27 |
| 5.51 | 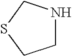 | ALDRICH | 570.54 | 3.17 |
| 5.52 | 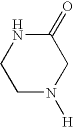 | ACROS | 581.50 | 2.8 |
| 5.53 | 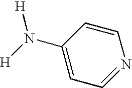 | ALDRICH | 575.5 | 2.76 |
| 5.54 | 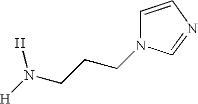 | ACROS | 606.56 | 2.7 |
| 5.55 | 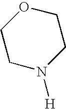 | ACROS | 568.51 | 3.08 |

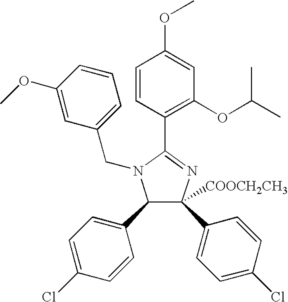

### Example 6

### trans-4,5-Bis-(4-chloro-phenyl)-2-(2-isopropoxy-4-methoxy-phenyl)-1-(3-methoxy-benzyl)-4,5-dihydro-1H-imidazole-4-carboxylic acid ethyl ester

To a solution of trans-4,5-bis-(4-chloro-phenyl)-2-(2-isopropoxy-4-methoxy-phenyl)-4,5-dihydro-1H-imidazole-4-carboxylic acid ethyl ester (Example 1, 25 mg, 0.047 mmol) in N,N-dimethylformamide (0.5 ml) are sequentially added cesium carbonate (31.2 mg, 0.0948 mmol) and 1-bromomethyl-3-methoxy-benzene (15.3 mg, 1.2 mmol). The reaction mixture is violently stirred overnight at room temperature. After filtration on a glass fritter, the solution is directly purified using a preparative LC-MS system (System 5) to give the desired compound as a trifluoroacetate salt, LC-MS (System 4) ([M+H]⁺=647.23, retention time=3.60 min).

### Example 7

### N-Alkylated Derivatives

In accordance with the procedures described in Example 6, the following compounds are prepared.

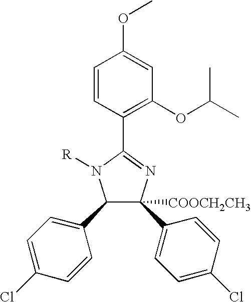

| | | | MW of the final | Retention time |
|---|---|---|---|---|
| | | | compounds as | (min) of LC-MS |
| Ex. | Bromides RBr | Supplier | free bases | (System 4) |
| 7.1 | 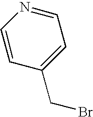 | ALDRICH | 618.56 | 2.82 |
| 7.4 | 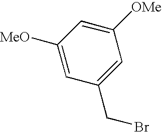 | ALDRICH | 677.63 | 3.50 |
| 7.9 | 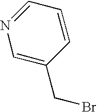 | ALDRICH | 618.56 | 2.94 |

### Example 8

### 1-Acyl-trans-4,5-dihydro imidazoles

To an array of glass tubes is added one of 55 carboxylic acids RCOOH (0.08 mmol) in each tube. 200 μL of a solution of trans-4,5-bis-(4-chloro-phenyl)-2-(4-methoxy-phenyl)-4,5-dihydro-1H-imidazole-4-carboxylic acid ethyl ester (1.44 g, 2.73 mmol) in 19.2 ml of N,N-dimethylformamide is added to each tube, followed by the addition in each tube of triethylamine (11.8 μL, 0.085 mmol). A 50% solution in N,N-dimethylformamide of propylphosphonic anhydride (54 μL, 0.085 mmol) is slowly added at room temperature to the reaction mixture. All tubes are closed by a aluminium cap and allowed to react at room temperature for 80 hours under continuous orbital shaking at 300 rpm. The array is then incubated at 50° C. for 17 hours and an additional 48 hours at 65° C. Methanol (0.8 ml) is added to each tube and the reaction mixtures are individually filtered over a 0.45 μm PTFA membrane. The filtrates are then purified by a preparative LC-MS procedure (System 8).

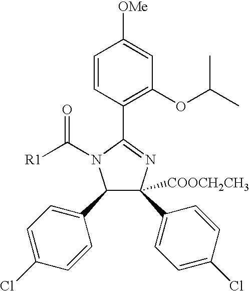

| | | | MW of the | Retention |
|---|---|---|---|---|
| | | | final | time (min) |
| | | | compounds | of LC-MS |
| | Carboxylic acids | | as free | (System |
| n° | R₁COOH | Supplier | bases | 9) |
| 8.1 | 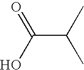 | ACROS | 597.54 | 3.83 |
| 8.2 | 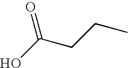 | FLUKA | 597.54 | 3.70 |
| 8.3 | 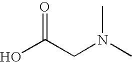 | ACROS | 612.56 | 2.82 |
| 8.4 | 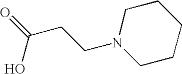 | ALDRICH | 666.65 | 2.91 |
| 8.5 | 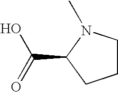 | SIGMA | 638.60 | 3.41 |
| 8.6 | 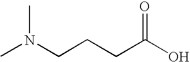 | ALDRICH | 640.61 | 2.75 |
| 8.7 | 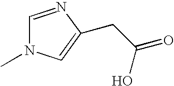 | SIGMA | 649.58 | 2.80 |
| 8.8 | 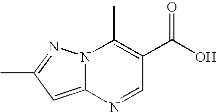 | MAYBRIDGE | 700.63 | 3.70 |
| 8.9 | 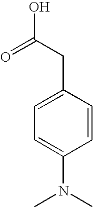 | ALDRICH | 688.66 | 3.70 |
| 8.10^{(a)} | 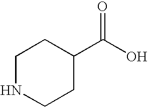 | MAYBRIDGE | 638.60 | 2.85 |
| 8.11 | 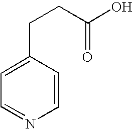 | CHEMBRIDGE CORPORATION | 660.60 | 3.04 |
| 8.12 | 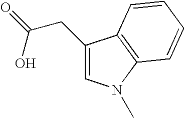 | ALDRICH | 698.65 | 3.90 |
| 8.13 | 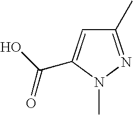 | MAYBRIDGE | 649.58 | 3.72 |
| 8.14 | 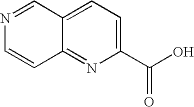 | APOLLO | 683.60 | 3.61 |
| 8.15 | 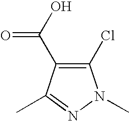 | MAYBRIDGE | 684.02 | 3.67 |
| 8.16 | 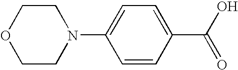 | MAYBRIDGE | 716.67 | 3.69 |
| 8.17^{(a)} | 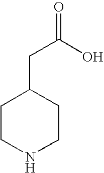 | FLUKA | 652.62 | 2.87 |
| 8.18 | 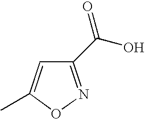 | MAYBRIDGE | 636.54 | 3.76 |
| 8.19 | 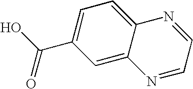 | MAYBRIDGE | 683.60 | 3.65 |
| 8.20 | 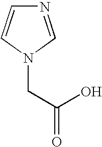 | MAYBRIDGE | 635.55 | 2.84 |
| 8.21^{(a)} | 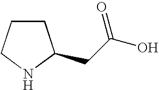 | FLUKA | 638.60 | 2.86 |
| 8.22 | 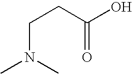 | TCI | 626.59 | 2.85 |
| 8.23 | 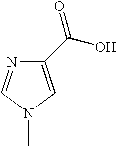 | MAYBRIDGE | 635.55 | 3.35 |
| 8.24 | 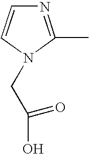 | MATRIX | 649.58 | 2.79 |
| 8.25 | 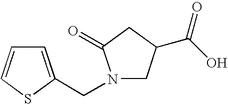 | MAYBRIDGE | 734.71 | 3.67 |
| 8.26 | 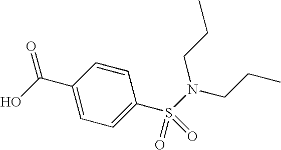 | MAYBRIDGE | 794.80 | 3.96 |
| 8.27^{(a)} | 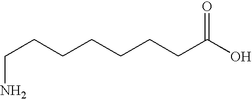 | FLUKA | 668.67 | 2.89 |
| 8.28 | 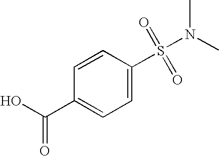 | ENAMINE | 738.69 | 3.74 |
| 8.29 | 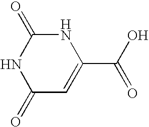 | FLUKA | 665.54 | 3.36 |
| 8.30 | 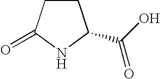 | FLUKA | 638.55 | 3.41 |
| 8.31^{(a)} | 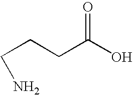 | BACHEM | 612.56 | 2.79 |
| 8.32 | 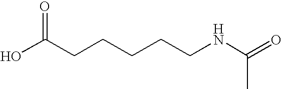 | ALDRICH | 682.65 | 3.49 |
| 8.33 | 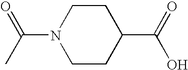 | LANCASTER | 680.63 | 3.52 |
| 8.34 | 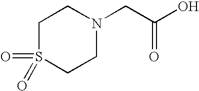 | TCI | 702.66 | 3.56 |
| 8.35 | 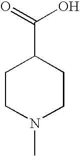 | EMKA-CHEMIE | 652.62 | 2.82 |
| 8.36 | 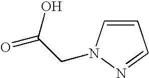 | ASINEX | 635.55 | 3.65 |
| 8.37 | 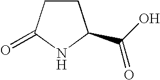 | FLUKA | 638.55 | 3.41 3.44 |

^{(a)}The corresponding N-BOC protected carboxylic acid is used for synthesis
N-BOC deprotection procedure: Fractions containing the desired N-BOC protected compound are pooled and solvents are evaporated. A mixture of trifluoroacetic acid (400 μl), dichloromethane (500 μl) and water (100 μl) is added. The reaction mixture is allowed to stand at room temperature for 17 hours before removal of the solvents

### Example 9

### Trans-4,5-Bis-(4-chloro-phenyl)-1-ethenesulfonyl-2-(2-isopropoxy-4-methoxy-phenyl)-4,5-dihydro-1H-imidazole-4-carboxylic acid ethyl ester

Triethylamine (712.5 μL, 5.12 mmol) and 2-chloroethanesulfonyl chloride (549 μL, 5.12 mmol) are successively added to a solution of trans-4,5-bis-(4-chloro-phenyl)-2-(2-isopropoxy-4-methoxy-phenyl)-4,5-dihydro-1H-imidazole-4-carboxylic acid ethyl ester (Step 2.1, 450 mg, 0.853 mmol) in dichloromethane (13 mL). The reaction is stirred overnight at room temperature. The residue obtained after evaporation is purified by an Isco Combiflash® Companion™ flash chromatography system on normal phase (40 g SiO₂, flow rate 40 mL/min) with a linear gradient: hexane/tert-butyl methyl ether 50:50 for two minutes followed by 16 minutes elution 50:50 to 100% tert-butyl methyl ether. The product is isolated as a white solid and identified by LC-MS (system 4) ([M+H]⁺=617.06, retention time=3.84 min).

### Example 10

### Trans-4,5-Bis-(4-chloro-phenyl)-2-(2-isopropoxy-4-methoxy-phenyl)-1-(2-morpholin-4-yl-ethanesulfonyl)-4,5-dihydro-1H-imidazole-4-carboxylic acid ethyl ester

To a solution of trans-4,5-bis-(4-chloro-phenyl)-1-ethenesulfonyl-2-(2-isopropoxy-4-methoxy-phenyl)-4,5-dihydro-1H-imidazole-4-carboxylic acid ethyl ester (Example 11, 18 mg, 0.029 mmol) in toluene (700 μL) is added morpholine (9 μL, 0.1 mmol). The reaction is carried out under sealed-vessel microwave heating at 120° C. for 10 minutes using a Biotage Initiator™ (pre-stirring: 15 s, absorption level: normal, microwave vial size 0.5-2 mL). The residue obtained after evaporation is purified by an Isco Combiflash® Companion™ flash chromatography system on normal phase (4 g SiO₂, flow rate 18 mL/min) with linear gradient: dichloromethane 100% for 2 min followed by 13 min elution to dichloromethane/tert-butyl methyl ether 50:50 and then 5 min elution dichloromethane/tert-butyl methyl ether 50:50. The product is isolated as a white solid and identified by LC-MS (system 4) ([M+H]⁺=704.22, retention time=3.30 min).

### Example 11

### Trans-4,5-Bis-(4-chloro-phenyl)-1-{2-[4-(2-hydroxy-ethyl)-piperazin-1-yl]-ethanesulfonyl}-2-(2-isopropoxy-4-methoxy-phenyl)-4,5-dihydro-1H-imidazole-4-carboxylic acid ethyl ester

To a solution of trans-4,5-bis-(4-chloro-phenyl)-1-ethenesulfonyl-2-(2-isopropoxy-4-methoxy-phenyl)-4,5-dihydro-1H-imidazole-4-carboxylic acid ethyl ester (Example 11, 30 mg, 0.049 mmol) in toluene (700 μL) is added hydroxyethylpiperazine (20.9 μL, 0.17 mmol). The reaction is carried out under sealed-vessel microwave heating at 120° C. for 10 minutes using a Biotage Initiator™ (pre-stirring: 15 s, absorption level: normal, microwave vial size 0.5-2 mL). The residue obtained after evaporation is purified by an Isco Combiflash® Companion™ flash chromatography system on normal phase (4 g SiO₂, flow rate 18 mL/min) with linear gradient: dichloromethane 100% for 2 min followed by 13 min elution to dichloromethane/tert-butyl methyl ether 50:50 and then 5 min elution dichloromethane/tert-butyl methyl ether 50:50. The product is isolated as a white solid and identified by LC-MS (system 4) ([M+H]⁺=747.30, retention time=3.17 min).

### Example 12

### Trans-4,5-Bis-(4-chloro-phenyl)-2-(2-isopropoxy-4-methoxy-phenyl)-1-[2-(3-oxo-piperazin-1-yl)-ethanesulfonyl]-4,5-dihydro-1H-imidazole-4-carboxylic acid ethyl ester

To a solution of trans-4,5-bis-(4-chloro-phenyl)-1-ethenesulfonyl-2-(2-isopropoxy-4-methoxy-phenyl)-4,5-dihydro-1H-imidazole-4-carboxylic acid ethyl ester (Example 11, 30 mg, 0.049 mmol) in toluene (700 μL) is added 2-oxo-piperazine (17 mg, 0.17 mmol). The reaction is carried out under sealed-vessel microwave heating at 120° C. for 10 minutes using a Biotage Initiator™ (pre-stirring: 15 s, absorption level: normal, microwave vial size 0.5-2 mL). The residue obtained after evaporation is purified by an Isco Combiflash® Companion™ flash chromatography system on normal phase (4 g SiO₂, flow rate 18 mL/min) with linear gradient: dichloromethane 100% for 2 min followed by 20 min elution to dichloromethane/methanol 90:10. The product is isolated as a white solid and identified by LC-MS (system 4) ([M+H⁺=717.21, retention time=3.40 min).

### Example 13

### Trans-4,5-Bis-(4-chloro-phenyl)-2-(2-isopropoxy-4-methoxy-phenyl)-1-[2-(4-methyl-piperazin-1-yl)-ethanesulfonyl]-4,5-dihydro-1H-imidazole-4-carboxylic acid ethyl ester

To a solution of trans-4,5-bis-(4-chloro-phenyl)-1-ethenesulfonyl-2-(2-isopropoxy-4-methoxy-phenyl)-4,5-dihydro-1H-imidazole-4-carboxylic acid ethyl ester (Example 11, 30 mg, 0.049 mmol) in toluene (700 μL) is added N-methyl-piperazine (18.9 μL, 0.17 mmol). The reaction is carried out under sealed-vessel microwave heating at 120° C. for 10 minutes using a Biotage Initiator™ (pre-stirring: 15 s, absorption level: normal, microwave vial size 0.5-2 mL). The residue obtained after evaporation is purified by an Isco Combiflash® Companion™ flash chromatography system on normal phase (4 g SiO₂, flow rate 18 mL/min) with linear gradient: dichloromethane 100% for 2 min followed by 18 min elution to dichloromethane/methanol 80:20. The product is isolated as a white solid and identified by LC-MS (system 4) ([M+H]⁺=717.25, retention time=3.22 min).

### Example 14

### Trans-4,5-Bis-(4-chloro-phenyl)-1-(2-diethylamino-ethanesulfonyl)-2-(2-isopropoxy-4-methoxy-phenyl)-4,5-dihydro-1H-imidazole-4-carboxylic acid ethyl ester

To a solution of trans-4,5-bis-(4-chloro-phenyl)-1-ethenesulfonyl-2-(2-isopropoxy-4-methoxy-phenyl)-4,5-dihydro-1H-imidazole-4-carboxylic acid ethyl ester (Example 11, 30 mg, 0.049 mmol) in toluene (700 μL) is added N,N-diethylamine (17.7 μL, 0.17 mmol). The reaction is carried out under sealed-vessel microwave heating at 120° C. for 10 minutes using a Biotage Initiator™ (pre-stirring: 15 s, absorption level: normal, microwave vial size 0.5-2 mL). The residue obtained after evaporation is purified by an Isco Combiflash® Companion™ flash chromatography system on normal phase (4 g SiO₂, flow rate 18 mL/min) with linear gradient: dichloromethane 100% for 2 min followed by 20 min elution to dichloromethane/tert-butyl methyl ether 45:55. The product is isolated as a white solid and identified by LC-MS (system 4) ([M+H]⁺=690.24, retention time=3.35 min).

### Example 15

### Trans-4,5-Bis-(4-chloro-phenyl)-1-isobutyl-2-(2-isopropoxy-4-methoxy-phenyl)-4,5-dihydro-3H-imidazole-4-carboxylic acid

To a solution of trans-4,5-bis-(4-chloro-phenyl)-2-(2-isopropoxy-4-methoxy-phenyl)-4,5-dihydro-1H-imidazole-4-carboxylic acid ethyl ester (Step 2.1, 50 mg, 0.095 mmol) in N,N-dimethylformamide (0.6 ml) is sequentially added cesium carbonate (62.4 mg, 0.190 mmol), isobutyl bromide (15.5 ul, 0.142 mmol) and tetrabutylammonium iodide (3.50 mg, 0.00948 mmol). The reaction is carried out under sealed-vessel heating at 80° C. overnight. Filtered on a glass fritter, the solution obtained is directly purified using a preparative LC-MS system (system 5)) to give the desired compound, LC-MS (system 4) ([M+H]⁺=555.12, retention time=3.46 min).

### Example 16

### Trans-4,5-Bis-(4-chloro-phenyl)-5-ethoxycarbonyl-2-(2-isopropoxy-4-methoxy-phenyl)-1,3-dimethyl-4,5-dihydro-3H-imidazole-1-ium

To a solution of trans-4,5-Bis-(4-chloro-phenyl)-2-(2-isopropoxy-4-methoxy-phenyl)-4,5-dihydro-1H-imidazole-4-carboxylic acid ethyl ester (50 mg, 0.095 mmol) in nitromethane (0.4 ml) is sequentially added barium oxide (18.9 mg, 0.123 mmol) and iodomethane (29.5 ul, 0.474 mmol). The reaction is carried out under argon and under sealed-vessel heating at 80° C. for 4 hours. Filtered on a glass fritter, the solution obtained is directly purified using a preparative LC-MS system (system 5) to give the desired compound, LC-MS (system 4) ([M+H]⁺=555.07, retention time=3.50 min).

### Example 17

### Soft Capsules

5000 soft gelatin capsules, each comprising as active ingredient 0.05 g of one of the compounds of formula I mentioned in the preceding Examples, are prepared as follows:

Composition

| | |
|---|---|
| Active ingredient | 250 g |
| Lauroglycol | 2 litres |

Preparation process: The pulverized active ingredient is suspended in Lauroglykol® (propylene glycol laurate, Gattefossé S. A., Saint Priest, France) and ground in a wet pulverizer to produce a particle size of about 1 to 3 μm. 0.419 g portions of the mixture are then introduced into soft gelatin capsules using a capsule-filling machine.

1. A dihydroimidazole of formula rac-(I), 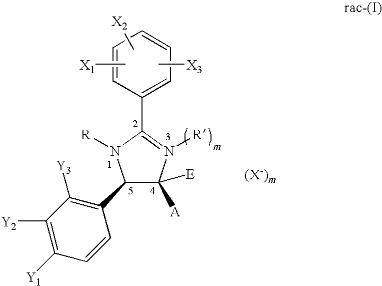
wherein
R is
(a) —C(O)R₁, wherein
R₁ denotes NRaRb, wherein Ra and Rb independently of each other denote hydrogen,
unsubstituted or substituted C1-C10 alkyl;
an unsubstituted or substituted monocyclic or bicyclic ring system comprising five to ten carbon atoms and being partially saturated or fully partially saturated; or
an unsubstituted or substituted monocyclic ring system being fully saturated or fully unsaturated and containing between two and five carbon atoms and one or two hetero atoms selected from N and O; or
wherein Ra and Rb together with the nitrogen atom to which they are attached represent an unsubstituted or substituted monocyclic ring system being fully saturated or partially saturated and containing between two and five carbon atoms and one or two hetero atoms selected from N and O; or
R₁ denotes unsubstituted or substituted C1-C8 alkyl, unsubstituted or substituted C3-C4-cycloalkyl, unsubstituted or substituted aryl or an unsubstituted or substituted mono- or bicyclic ring system being fully unsaturated, partially saturated or fully saturated and containing between two and eight carbon atoms and one, two or three hetero atoms selected from N, S and O;
(b) C1-C6 alkyl, which is unsubstituted or substituted by unsubstituted or substituted aryl or a mono- or bicyclic ring system being fully unsaturated and containing between two and six carbon atoms and one, two or three hetero atoms selected from N and O;
(c) C3-C5 alkenyl;
(d) —SO₂—R₆, wherein R₈ denotes unsubstituted or substituted C1-C4 alkyl, C3-C5 alkenyl, or unsubstituted or substituted aryl; or
(e) hydrogen;
R′ denotes C1-C6 alkyl,
m is 0 or 1 under the proviso that if m is 1, the nitrogen atom to which R′ is attached is positively charged,
X⁻ is an anion derived from an organic or inorganic acid,
X₁, X₂ and X₃ are independently selected from —OH, C1-C2 alkyl, C1-C6 alkoxy, Cl, Br, F, —CH₂OCH₃, and —CH₂OCH₂CH₃, or one of X₁, X₂ and X₃ is H and the other two are independently selected from hydroxy, C1-C6 alkyl, C1-C6 alkoxy, Cl, Br, F, CF₃, —CH₂OCH₃, —CH₂OCH₂CH₃, ‘OCH₂CH₂R₃, OCH₂CF₃, and —OR₁, or one of X₁, X₂ and X₃ is H and the other two taken together with the two carbon atoms to which they are attached form a 5- or 6-membered saturated ring that contains at least one hetero atom selected from S, N and O, wherein
R₃ is selected from F, —OCH₃, —N(CH₃)CH₃, Cl, Br, an unsaturated 5- and 6 membered ring containing at least one hetero atom wherein the hetero atom is selected from N and O, and
R₄ is C3-C5 cycloalkyl;
Y₁ denotes halo, NO₂, CN, or —CCH;
Y₂ denotes hydrogen or halo;
Y₃ denotes hydrogen or hydroxy;
A is alkyl, cycloalkyl being unsubstituted or substituted by alkyl, or a radical of subformula (Ia), 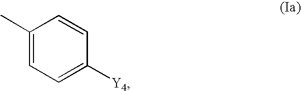
wherein Y₄ denotes halo, NO₂, CN, or —CCH; and
E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or
C(O)Z, wherein
Z represents
hydroxy, C1-C4 alkoxy, amino which is mono- or disubstituted by unsubstituted or substituted C1-C6 alkyl, or an unsubstituted or substituted mono- or bicyclic ring system being fully unsaturated, partially saturated or fully saturated and containing between two and eight carbon atoms and one, two or three hetero atoms selected from N, S and O, such ring system;
or a tautomer thereof,
or a salt of such dihydroimidazole or its tautomer.

2. The dihydroimidazole of formula rac-(I) according to claim 1,
wherein
R is
(a) —C(O)R₁, wherein
R₁ denotes NRaRb, wherein Ra and Rb independently of each other denote hydrogen,
unsubstituted or substituted C1-C10 alkyl;
an unsubstituted or substituted monocyclic or bicyclic ring system comprising five to ten carbon atoms and being partially saturated or fully partially saturated; or
an unsubstituted or substituted monocyclic ring system being fully saturated or fully unsaturated and containing between two and five carbon atoms and one or two hetero atoms selected from N and O; or
wherein Ra and Rb together with the nitrogen atom to which they are attached represent an unsubstituted or substituted monocyclic ring system being fully saturated or partially saturated and containing between two and five carbon atoms and one or two hetero atoms selected from N and O; or
R₁ denotes unsubstituted or substituted C1-C8 alkyl, unsubstituted or substituted C3-C4-cycloalkyl, unsubstituted or substituted aryl or an unsubstituted or substituted mono- or bicyclic ring system being fully unsaturated, partially saturated or fully saturated and containing between two and eight carbon atoms and one, two or three hetero atoms selected from N, S and O;
(b) C1-C4 alkyl, which is unsubstituted or substituted by unsubstituted or substituted aryl or a mono- or bicyclic ring system being fully unsaturated and containing between two and six carbon atoms and one, two or three hetero atoms selected from N and O;
(c) C3-C5 alkenyl;
(d) —SO₂—R₆, wherein R₆ denotes unsubstituted or substituted C1-C4 alkyl, C3-C5 alkenyl, or unsubstituted or substituted aryl; or
(e) hydrogen;
R′ denotes C1-C6 alkyl,
m is 0 or 1 under the proviso that if m is 1, the nitrogen atom to which R′ is attached is positively charged,
X⁻ is an anion derived from an organic or inorganic acid,
X₁, X₂ and X₃ are independently selected from —OH, C1-C2 alkyl, C1-C6 alkoxy, Cl, Br, F, —CH₂OCH₂CH₃, or one of X₁, X₂ and X₃ is H and the other two are independentlyselected from hydroxy, C1-C6 alkyl, C1-C6 alkoxy, Cl, Br, F, CF₃, —CH₂OCH₃, —CH₂OCH₂CH₃, —OCH₂CH₂R₃, OCH₂CF₃, and —OR₄, or one of X₁, X₂ and X₃ is H and the other two taken together with the two carbon atoms to which they are attached form a 5- or 6-membered saturated ring that contains at least one hetero atom selected from S, N and O, wherein
R₃ is selected from F, —OCH₃, —N(CH₃)CH₃, Cl, Br, an unsaturated 5- and 6 membered ring containing at least one hetero atom wherein the hetero atom is selected from N and O, and
R₄ is C3-C5 cycloalkyl;
Y₁ denotes halo, NO₂, CN, or —CCH;
Y₂ and Y₃ denote hydrogen;
A is a radical of subformula (Ia), wherein Y₄ denotes halo, NO₂, CN, or —CCH; and
E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or
C(O)Z, wherein
Z represents
hydroxy, C1-C4 alkoxy, amino which is mono- or disubstituted by unsubstituted or substituted C1-C6 alkyl, or an unsubstituted or substituted mono- or bicyclic ring system being fully unsaturated, partially saturated or fully saturated and containing between two and eight carbon atoms and one, two or three hetero atoms selected from N, S and O, such ring system;
or a tautomer thereof,
or a salt of such dihydroimidazole or its tautomer.

3. The dihydroimidazole of formula rac-(I) according to claim 1,
wherein
R is
(a) —C(O)R₁, wherein
R₁ denotes NRaRb,
wherein Ra and Rb independently of each other denote
C1-C10 alkyl which is unsubstituted or mono-, di- or trisubstituted by cyano, C1-C4 alkoxy which is unsubstituted or substituted by phenyl, C3-C6 cycloalkyl, hydroxy, carbamoyl, N—C1-C4 alkyl carbamoyl, di(C1-C4 alkyl)-amino; a monocyclic or bicyclic ring system being unsaturated, partially saturated or fully saturated and containing between two and ten carbon atoms and one or two hetero atoms selected from N, O and S, such ring system being unsubstituted or substituted by C1-C4 alkyl, C2-C4 alkanoyl or oxo; or phenyl, which is unsubstituted or mono- or disubstituted by halo, aminosulfonyl, or a monocyclic ring system being fully saturated and containing between two and five carbon atoms and one or two hetero atoms selected from N and O, such ring system being unsubstituted or substituted by C1-C4 alkyl;
hydrogen,
a monocyclic or bicyclic ring system comprising five to ten carbon atoms and being partially saturated or fully partially saturated and optionally substituted by hydroxy, hydroxy C1-C4 alkyl or carbamoyl; or
a monocyclic ring system being fully saturated or fully unsaturated and containing between two and five carbon atoms and one or two hetero atoms selected from N and O, such ring system being unsubstituted or substituted by C1-C4 alkyl; or
wherein Ra and Rb together with the nitrogen atom to which they are attached represent a monocyclic ring system being fully saturated or partially saturated and containing between two and five carbon atoms and one or two hetero atoms selected from N and O, such ring system being unsubstituted or mono- or disubstituted by hydroxy, C2-C4 alkanoyl, carbamoyl, C1-C4 alkoxy carbonyl, C1-C4 alkyl which is unsubstituted or substituted by hydroxy, di(C1-C4 alkyl)-amino, morpholinyl carbonyl, piperidinyl carbonyl or pyrrolidinyl carbonyl; pyrimidinyl, phenyl, C1-C4 alkyl piperidinyl or oxo; phenyl, pyrrolidinyl, (1H)-2,3-dihydro-2-oxo-benzimidazolyl, or
R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, a mono- or bicyclic ring system being fully unsaturated or fully saturated and containing between two and eight carbon atoms and one, two or three hetero atoms selected from N, S and O, such ring system being unsubstituted or substituted by C1-C4 alkyl or oxo; or by phenyl which is substituted by di(C1-C4 alkyl)-amino;
C3-C4-cycloalkyl which is unsubstituted or substituted by C1-C4 alkyl;
phenyl being substituted by morpholinyl, di(C1-C4 alkyl)-amino or di(C1-C4 alkyl)-amino-sulfonyl; or;
a mono- or bicyclic ring system being fully unsaturated, partially saturated or fully saturated and containing between two and eight carbon atoms and one, two or three hetero atoms selected from N, S and O, such ring system being unsubstituted or substituted by halo, oxo, C1-C4 alkyl, thienyl-C1-C4 alkyl, halophenyl-C1-C4 alkyl, C1-C4 alkyl carbonyl or phenyl;
(b) C1-C4 alkyl, which is substituted by
phenyl which is mono- or di-substituted by C1-C4 alkyl, trifluoromethyl, C1-C4 alkoxy, trifluoromethoxy or cyano; or
a mono- or bicyclic ring system being fully unsaturated and containing between two and six carbon atoms and one, two or three hetero atoms selected from N and O;
(c) C3-C5 alkenyl;
(d) —SO₂—R₆, wherein R₅ denotes
C1-C4 alkyl, which is substituted by di(C1-C4 alkyl)-amino, a monocyclic ring system being fully saturated and containing between four and five carbon atoms and two hetero atoms selected from N and O, such ring system being substituted by C1-C4 alkyl, hydroxy C1-C4 alkyl or oxo;
C3-C5 alkenyl;
naphthyl; or
phenyl, which is substituted by halo; or
(e) hydrogen;
R′ denotes C1-C6 alkyl,
m is 0 or 1 under the proviso that if m is 1, the nitrogen atom to which R′ is attached is positively charged,
X⁻ is an anion derived from an organic or inorganic acid,
X₁, X₂ and X₃ are independently selected from —OH, C1-C2 alkyl, C1-C6 alkoxy, Cl, Br, F, —CH₂OCH₃, and —CH₂OCH₂CH₃, or one of X₁, X₂ and X₃ is H and the other two are independently selected from hydroxy, C1-C6 alkyl, C1-C6 alkoxy, Cl, Br, F, CF₃, —CH₂OCH₃, —CH₂OCH₂CH₃, —OCH₂CH₂R₃, OCH₂CF₃, and —OR₄, or one of X₁, X₂ and X₃ is H and the other two taken together with the two carbon atoms to which they are attached form a 5- or 6-membered saturated ring that contains at least one hetero atom selected from S, N and O, wherein
R₃ is selected from F, —OCH₃, —N(CH₃)CH₃, Cl, Br, an unsaturated 5- and 6 membered ring containing at least one hetero atom wherein the hetero atom is selected from N and O, and
R₄ is a C3-C5 cycloalkyl;
Yₗ denotes Cl, Br, NO₂, CN, or —CCH;
Y₂ and Y₃ denote hydrogen;
A is a radical of subformula (Ia), wherein Y₄ denotes Cl, Br, NO₂, CN, or —CCH; and
E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C₁-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or
C(O)Z, wherein
Z represents
hydroxy or C1-C4 alkoxy,
amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, a mono- or bicyclic ring system being fully unsaturated, partially saturated or fully saturated and containing between two and eight carbon atoms and one, two or three hebro atoms selected from N, S and O, such ring system being unsubstituted or substituted by C1-C4 alkyl, C1-C4 alkyl carbonyl and oxo; or phenyl, which is unsubstituted or substituted by C1-C4 alkyl piperazinyl, halo or amino sulfonyl;
C3-C5 alkinyl; 1,2-dihydro-indanyl, phenyl or C4-C6 cycloalkyl; or
a mono- or bicyclic ring system being fully unsaturated, partially saturated or fully saturated and containing between two and eight carbon atoms and one, two or three hetero atoms selected from N, S and O, such ring system being unsubstituted or substituted by C1-C4 alkyl, di(C1-C4 alkylamino C1-C4 alkyl, C1-C4 alkyl carbonyl, C1-C4 alkyl piperidinyl, oxo; phenyl, pyrrolidinyl, amino carbonyl, C1-C4 alkoxy carbonyl or pyrimidyl;
or a tautomer thereof,
or a salt of such dihydroimidazole or its tautomer.

4. The dihydroimidazole of formula rac-(I) according to claim 1,
wherein
R is
(a) —C(O)R₁, wherein
R₁ denotes NRaRb,
wherein Ra and Rb independently of each other denote
C1-C10 alkyl which is unsubstituted or mono-, di- or trisubstituted by cyano, C1-C4 alkoxy which is unsubstituted or substituted by phenyl, C3-C6 cycloalkyl, hydroxy, carbamoyl, N—C1-C4 alkyl carbamoyl, di(C1-C4 alkyl)-amino; a monocyclic or bicyclic ring system being unsaturated, partially saturated or fully saturated and containing between two and ten carbon atoms and one or two hetero atoms selected from N, O and S, such ring system being unsubstituted or substituted by C1-C4 alkyl, C2-C4 alkanoyl or oxo; or phenyl, which is unsubstituted or mono- or disubstituted by halo, aminosulfonyl, or a monocyclic ring system being fully saturated and containing between two and five carbon atoms and one or two hetero atoms selected from N and O, such ring system being unsubstituted or substituted by C1-C4 alkyl;
hydrogen,
a monocyclic or bicyclic ring system comprising five to ten carbon atoms and being partially saturated or fully partially saturated and optionally substituted by hydroxy, hydroxy C1-C4 alkyl or carbamoyl; or
a monocyclic ring system being fully saturated or fully unsaturated and containing between two and five carbon atoms and one or two hetero atoms selected from N and O, such ring system being unsubstituted or substituted by C1-C4 alkyl; or
wherein Ra and Rb together with the nitrogen atom to which they are attached represent a monocyclic ring system being fully saturated or partially saturated and containing between two and five carbon atoms and one or two hetero atoms selected from N and O, such ring system being unsubstituted or mono- or disubstituted by hydroxy, C2-C4 alkanoyl, carbamoyl, C1-C4 alkoxy carbonyl, C1-C4 alkyl which is unsubstituted or substituted by hydroxy, di(C1-C4 alkyl)-amino, morpholinyl carbonyl, piperidinyl carbonyl or pyrrolidinyl carbonyl; pyrimidinyl, phenyl, C1-C4 alkyl piperidinyl or oxo; phenyl, pyrrolidinyl, (1H)-2,3-dihydro-2-oxo-benzimidazolyl, or
R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, a mono- or bicyclic ring system being fully unsaturated or fully saturated and containing between two and eight carbon atoms and one, two or three hetero atoms selected from N, S and O, such ring system being unsubstituted or substituted by C1-C4 alkyl or oxo; or by phenyl which is substituted by di(C1-C4 alkyl)-amino;
C3-C4-cycloalkyl which is unsubstituted or substituted by C1-C4 alkyl;
phenyl being substituted by morpholinyl, di(C1-C4 alkyl)-amino or di(C1-C4 alkyl)-amino-sulfonyl; or;
a mono- or bicyclic ring system being fully unsaturated, partially saturated or fully saturated and containing between two and eight carbon atoms and one, two or three hetero atoms selected from N, S and O, such ring system being unsubstituted or substituted by halo, oxo, C1-C4 alkyl, thienyl-C1-C4 alkyl, halophenyl-C1-C4 alkyl, C1-C4 alkyl carbonyl or phenyl;
(b) C1-C4 alkyl, which is substituted by
phenyl which is mono- or di-substituted by C1-C4 alkyl, trifluoromethyl, C1-C4 alkoxy, trifluoromethoxy or cyano; or - a mono- or bicyclic ring system being fully unsaturated and containing between two and six carbon atoms and one, two or three hetero atoms selected from N and O;
(c) C3-C5 alkenyl;
(d) —SO₂—R₆, wherein R₆ denotes
C1-C4 alkyl, which is substituted by di(C1-C4 alkyl)-amino, a monocyclic ring system being fully saturated and containing between four and five carbon atoms and two hetero atoms selected from N and O, such ring system being substituted by C1-C4 alkyl, hydroxy C1-C4 alkyl or oxo;
C3-C5 alkenyl;
naphthyl; or
phenyl, which is substituted by halo; or
(e) hydrogen;
R′ denotes C1-C6 alkyl,
m is 0 or 1 under the proviso that if m is 1, the nitrogen atom to which R′ is attached is positively charged,
X⁻ is an anion derived from an organic or inorganic acid,
X₁, X₂ and X₃ are independently selected from hydrogen and C1-C4 alkoxy,
Y₁ denotes halo;
Y₂ and Y₃ denote hydrogen;
A is a radical of subformula (Ia), wherein Y₄ denotes halo; and
E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or
C(O)Z, wherein
Z represents
C1-C4 alkoxy;
amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, a mono- or bicyclic ring system being fully unsaturated, partially saturated or fully saturated and containing between two and eight carbon atoms and one, two or three hetero atoms selected from N, S and O, such ring system being unsubstituted or substituted by C1-C4 alkyl, C1-C4 alkyl carbonyl and oxo; or phenyl, which is unsubstituted or substituted by C1-C4 alkyl piperazinyl, halo or amino sulfonyl;
C3-C5 alkinyl; 1,2-dihydro-indanyl, phenyl or C4-C6 cycloalkyl; or
a mono- or bicyclic ring system being fully unsaturated, partially saturated or fully saturated and containing between two and eight carbon atoms and one, two or three hero atoms selected from N, S and O, such ring system being unsubstituted or substituted by C1-C4 alkyl, di(C1-C4 alkyl)-amino C1-C4 alkyl, C1-C4 alkyl carbonyl, C1-C4 alkyl piperidinyl, oxo, phenyl, pyrrolidinyl, amino carbonyl, C1-C4 alkoxy carbonyl or pyrimidyl;
or a tautomer thereof,
or a salt of such dihydroimidazole or its tautomer.

5. The dihydroimidazole of formula rac-(I) according to claim 1, wherein
R is
(a) —C(O)R₁, wherein
R₁ denotes NRaRb,
wherein Ra and Rb independently of each other denote
C1-C10 alkyl which is unsubstituted or mono-, di- or trisubstituted by cyano, C1-C4 alkoxy which is unsubstituted or substituted by phenyl, hydroxy, carbamoyl, N—C1-C4 alkyl carbamoyl, di(C1-C4 alkyl)-amino, pyridyl, pyrrolyl, C1-C4 alkyl imidazolyl, furyl, indolyl, isochromanyl, benzothienyl; phenyl, which is unsubstituted or mono- or disubstituted by halo, morpholinyl, piperidinyl, aminosulfonyl, or C1-C4 alkyl piperazinyl; C3-C6 cycloalkyl; morpholinyl, tetrahydrofuranyl, tetrahydropyranyl, benzo[1,3]dioxolyl, C1-C4 alkyl piperazinyl, pyrrolidinyl which is unsubstituted or substituted by C1-C4 alkyl or oxo; C1-C4 alkyl-pyrazinyl, or piperidinyl, which is unsubstituted or substituted by C1-C4 alkyl or C2-C4 alkanoyl;
C5-C6 cycloalkyl which is unsubstituted or substituted by hydroxy, hydroxy C1-C4 alkyl or carbamoyl;
hydrogen,
indanyl, 2,3-dihydro-hydroxy-indanyl, 2,3-dihydro-2-indanyl,
C1-C4 alkyl-pyrazolyl or
piperidinyl substituted by C1-C4 alkyl; or
wherein Ra and Rb together with the nitrogen atom to which they are attached represent piperazine being unsubstituted or mono- or disubstituted by C2-C4 alkanoyl, C1-C4 alkyl which is unsubstituted or substituted by hydroxy, di(C1-C4 alkyl)-amino, morpholinyl carbonyl, piperidinyl carbonyl or pyrrolidinyl carbonyl; pyrimidinyl, phenyl, C1-C4 alkyl piperidinyl or oxo;
pyrrolidine being substituted by hydroxy C1-C4 alkyl,
piperidine being unsubstituted or substituted by phenyl, benzyl, pyrrolidinyl, (1H)-2,3-dihydro-2-oxo-benzimidazolyl, carbamoyl, C1-C4 alkoxy carbonyl, hydroxy or hydroxy C1-C4 alkyl;
tetrahydro-pyrimidine;
C1-C4 alkyl-1,4-diaza-cycloheptane, phenyl C1-C4 alkyl-1,4-diaza-cycloheptane, or morpholine; or
R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, imidazolyl, C1-C4 alkyl-imidazolyl, pyrazolyl, piperidinyl, pyrrolidinyl, 1,1-dioxo-thiomorpholinyl, pyridyl, indolyl, N—C1-C4 alkyl-indolyl; or phenyl which is substituted by di(C1-C4 alkyl)-amino;
C3-C4-cycloalkyl which is unsubstituted or substituted by C1-C4 alkyl;
phenyl being substituted by morpholinyl, di(C1-C4 alkyl)-amino or di(C1-C4 alkyl)-amino-sulfonyl;
N—C1-C4 alkyl-indolyl; N—C1-C4 alkyl-imidazolyl; quinoxalinyl; pyrazolo[1,5-a]pyrimidinyl being mono- or disubstituted by C1-C4 alkyl; [1,6]naphthyridinyl; oxazolyl being substituted by phenyl or C1-C4 alkyl; pyrazolyl being mono-, di- or trisubstituted by C1-C4 alkyl and halo;
piperidinyl, being unsubstituted or substituted by C1-C4 alkyl or C1-C4 alkyl carbonyl;
tetrahydropyrimidyl, disubstituted by oxo, or pyrrolidinyl which is mono- or disubstituted by radicals independently selected from the group consisting of C1-C4 alkyl, thienyl-C1-C4 alkyl, chlorophenyl-C1-C4 alkyl and oxo;
(b) C1-C4 alkyl, which is substituted by
phenyl which is mono- or di-substituted by C1-C4 alkyl, trifluoromethyl, C1-C4 alkoxy, trifluoromethoxy or cyano;
pyridyl; or
benzo[c]-1-oxa-2,5-diazolyl;
(c) C3-C5 alkenyl;
(d) —SO₂—R₆, wherein R₆ denotes
C1-C4 alkyl, which is substituted by di(C1-C4 alkyl)-amino, morpholinyl or piperazinyl which is further substituted by C1-C4 alkyl, hydroxy C1-C4 alkyl or oxo;
C3-C5 alkenyl;
naphthyl; or
phenyl, which is substituted by halo; or
(e) hydrogen;
R′ denotes C1-C6 alkyl,
m is 0 or 1 under the proviso that if m is 1, the nitrogen atom to which R′ is attached is positively charged,
X⁻ is an anion derived from an organic or inorganic acid,
X₁, X₂ and X₃ are independently selected from hydrogen and C1-C4 alkoxy,
Y₁ denotes halo;
Y₂ and Y₃ denote hydrogen;
A is a radical of subformula (Ia), wherein Y₄ denotes halo; and
E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C1-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl; or C(O)Z, wherein
Z represents
C1-C4 alkoxy;
amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, pyridyl, pyrrolyl, imidazolyl, C1-C4 alkyl imidazolyl, C1-C4 alkyl pyrimidyl, C1-C4 alkyl pyrazinyl, furyl, dihydroisochromanyl, tetrahydro-pyranyl, tetrahydrofuryl, morpholinyl, pyrrolidinyl, C1-C4 alkyl pyrrolidinyl, 2-oxo-pyrrolidinyl, piperidinyl, C1-C4 alkyl carbonyl piperidinyl, C1-C4 alkyl piperidinyl, C1-C4 alkyl piperazinyl; or phenyl, which is unsubstituted or substituted by C1-C4 alkyl piperazinyl, halo or amino sulfonyl; or C3-C5 alkinyl; pyridyl, thiazolyl, C1-C4 alkyl oxazolyl, isoxazolyl: pyrazinyl, pyrimidyl, C1-C4 alkyl pyrazolyl, 1,2-dihydro-indanyl, phenyl or C4-C6 cycloalkyl;
pyrrolidinyl;
piperazinyl, which is unsubstituted or substituted by C1-C4 alkyl, di(C1-C4 alkyl)-amino C1-C4 alkyl, C1-C4 alkyl carbonyl, C1-C4 alkyl piperidinyl, oxo or pyrimidyl;
piperidinyl, which is unsubstituted or substituted by phenyl, pyrrolidinyl, amino carbonyl or C1-C4 alkoxy carbonyl;
morpholinyl;
tetrahydrothiazolyl; or
C1-C4 alkyl-1,4-diaza-cycloheptane;
or a tautomer thereof,
or a salt of such dihydroimidazole or its tautomer.

6. The dihydroimidazole of formula rac-(I) according to claim 1, wherein
R is
(a) —C(O)R₁, wherein
R₁ denotes NRaRb,
wherein Ra and Rb independently of each other denote
C1-C10 alkyl which is unsubstituted or mono-, di- or trisubstituted by cyano, C1-C4 alkoxy which is unsubstituted or substituted by phenyl, hydroxy, carbamoyl, N-methyl carbamoyl, di(C1-C4 alkyl)-amino, pyridyl, 1-pyrrolyl, C1-C4 alkyl imidazolyl, furyl, 3-indolyl, isochromanyl, benzothienyl; phenyl, which is unsubstituted or mono- or disubstituted by chloro, morpholinyl, piperidinyl, aminosulfonyl, or C1-C4 alkyl piperazinyl; C3-C6 cycloalkyl; morpholin-4-yl, tetrahydrofuranyl, tetrahydropyranyl, benzo[1,3]dioxolyl, C1-C4 alkyl piperazinyl, pyrrolidinyl which is unsubstituted or substituted by C1-C4 alkyl or oxo; C1-C4 alkyl-pyrazinyl, or piperidinyl, which is unsubstituted or substituted by C1-C4 alkyl or C2-C4 alkanoyl;
C5-C6 cycloalkyl which is unsubstituted or substituted by hydroxy, hydroxy C1-C4 alkyl or carbamoyl;
hydrogen,
indanyl, 2,3-dihydro-2-hydroxy-indanyl, 2,3-dihydro-2-indanyl,
1-C1-C4 alkyl-pyrazolyl or
4-piperidinyl substituted by C1-C4 alkyl; or
wherein Ra and Rb together with the nitrogen atom to which they are attached represent piperazine being unsubstituted or mono- or disubstituted by C2-C4 alkanoyl, C1-C4 alkyl which is unsubstituted or substituted by hydroxy, di(C1-C4 alkyl)-amino, morpholinyl carbonyl, piperidinyl carbonyl or 1-pyrrolidinyl carbonyl; 2-pyrimidinyl, phenyl, C1-C4 alkyl piperidinyl or oxo;
pyrrolidine being substituted by hydroxy C1-C4 alkyl,
piperidine being unsubstituted or substituted by phenyl, benzyl, 1-pyrrolidinyl, (1H)-2,3-dihydro-2-oxo-benzimidazol-1-yl, carbamoyl, C1-C4 alkoxy carbonyl, hydroxy or hydroxy C1-C4 alkyl;
tetrahydro-pyrimidine;
4-C1-C4 alkyl-1,4-diaza-cycloheptane, 4-benzyl-1,4-diaza-cycloheptane, or morpholine; or
R₁ denotes
C1-C8 alkyl which is unsubstituted or substituted by amino, C1-C4 alkyl carbonyl amino, C1-C4 alkyl-phenyl carbonyl amino, di(C1-C4 alkyl)-amino, imidazolyl, C1-C4 alkyl-imidazolyl, pyrazolyl, piperidinyl, pyrrolidinyl, 1,1-dioxo-thiomorpholinyl, pyridyl, indolyl, N—C1-C4 alkyl-indolyl; or phenyl which is substituted by di(C1-C4 alkyl)-amino;
C3-C4-cycloalkyl which is unsubstituted or substituted by C1-C4 alkyl;
phenyl being substituted by morpholinyl, di(C1-C4 alkyl)-amino or di(C1-C4 alkyl)-amino-sulfonyl;
N—C1-C4 alkyl-indolyl; N—C1-C4 alkyl-imidazolyl; quinoxalinyl; pyrazolo[1,5-a]pyrimidinyl being disubstituted by C1-C4 alkyl; [1,6]naphthyridinyl; oxazolyl being substituted by phenyl or C1-C4 alkyl; pyrazolyl being di- or trisubstituted by C1-C4 alkyl and chloro; piperidinyl, being unsubstituted or substituted by C1-C4 alkyl or C1-C4 alkyl carbonyl; tetrahydropyrimidyl, disubstituted by oxo, or pyrrolidinyl which is mono- or disubstituted by radicals independently selected from the group consisting of C1-C4 alkyl, thienyl-C1-C4 alkyl, chlorophenyl-C1-C4 alkyl and oxo;
(b) C1-C4 alkyl, which is substituted by
phenyl which is mono- or di-substituted by C1-C4 alkyl, trifluoromethyl, C1-C4 alkoxy,
trifluoromethoxy or cyano;
pyridyl; or
benzo[c]-1-oxa-2,5-diazolyl;
(c) C3-C4 alkenyl;
(d) SO₂—R₆, wherein R₆ denotes
C1-C4 alkyl, which is substituted by di(C1-C4 alkyl)-amino, morpholinyl or piperazinyl which is further substituted by C1-C4 alkyl, hydroxy C1-C4 alkyl or oxo;
C3-C4 alkenyl;
naphthyl; or
phenyl, which is substituted by fluoro; or
(e) hydrogen;
R′ denotes C1-C6 alkyl,
m is 0 or 1 under the proviso that if m is 1, the nitrogen atom to which R′ is attached is positively charged,
X⁻ is an anion derived from an organic or inorganic mid,
X₁, X₂ and X₃ are independently selected from hydrogen and C1-C4 alkoxy,
Y₁ denotes halo;
Y₂ and Y₃ denote hydrogen;
A is a radical of subformula (Ia), wherein Y₄ denotes halo; and
E denotes halo, cyano, hydroxy, mercapto, alkylthio, phenylthio, B(OH)₂, formyl, carboxy, C1-C4 alkoxy, C1-C4 alkyl, C2-C5 alkenyl, C2-C5 alkanoyl, hydroxy C1-C4 alkyl, di-C₁-C4 alkyl amino alkyl, aryl, aryloxy, hetaryl, amino, C1-C4 alkyl amino, di-C1-C4 alkyl amino, aryl amino, aryl (C1-C4 alkyl) amino, C(O)C(O) C1-C4 alkoxy, C(S)N(H) aryl;
or C(O)Z, wherein
Z represents
C1-C4 alkoxy; C1-C4 alkyl;
amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, pyridyl, pyrrolyl, imidazolyl, C1-C4 alkyl imidazolyl, C1-C4 alkyl pyrimidyl, C1-C4 alkyl pyrazinyl, furyl, dihydro-isochromanyl, tetrahydro-pyranyl, tetrahydrofuryl, morpholinyl, pyrrolidinyl, C1-C4 alkyl pyrrolidinyl, 2-oxo-pyrrolidinyl, piperidinyl, C1-C4 alkyl carbonyl piperidinyl, C1-C4 alkyl piperidinyl, C1-C4 alkyl piperazinyl; or phenyl, which is unsubstituted or substituted by C1-C4 alkyl piperazinyl, fluoro or amino sulfonyl; or C3-C5 alkinyl, pyridyl, thiazolyl, C1-C4 alkyl oxazolyl, isoxazolyl, pyrazinyl, pyrimidyl, C1-C4 alkyl pyrazolyl, 1,2-dihydro-indanyl, phenyl or C4-C6 cycloalkyl;
pyrrolidinyl;
piperazinyl, which is unsubstituted or substituted by C1-C4 alkyl, di(C1-C4 alkyl)-amino C1-C4 alkyl, C1-C4 alkyl carbonyl, C1-C4 alkyl piperidinyl, oxo or pyrimidyl;
piperidinyl, which is unsubstituted or substituted by phenyl, pyrrolidinyl, amino carbonyl or C1-C4 alkoxy carbonyl;
morpholinyl;
tetrahydrothiazolyl; or
4-C1-C4 alkyl-1,4-diaza-cycloheptane;
or a tautomer thereof,
or a salt of such dihydroimidazole or its tautomer.

7. The dihydroimidazole of formula rac-(I) according to claim 1, wherein the absolute configuration of C-5 in the dihydroimidazole ring is “R”.

8. The dihydroimidazole of formula rac-(I) according to claim 1, wherein
E denotes C(O)Z and
Z represent
C1-C4 alkoxy;
amino which is mono- or disubstituted by C1-C6 alkyl which is unsubstituted or substituted by fluoro, di(C1-C4 alkyl)-amino, amino carbonyl, C1-C4 alkyl amino carbonyl, C1-C4 alkyl carbonyl amino, C1-C4 alkoxy, C1-C4 alkoxy carbonyl, cyano, C3-C6 cycloalkyl, pyridyl, pyrrolyl, imidazolyl, C1-C4 alkyl imidazolyl, C1-C4 alkyl pyrimidyl, C1-C4 alkyl pyrazinyl, furyl, dihydro-isochromanyl, tetrahydro-pyranyl, tetrahydrofuryl, morpholinyl, pyrrolidinyl, C1-C4 alkyl pyrrolidinyl, 2-oxo-pyrrolidinyl, piperidinyl, C1-C4 alkyl carbonyl piperidinyl, C1-C4 alkyl piperidinyl, C1-C4 alkyl piperazinyl; or phenyl, which is unsubstituted or substituted by C1-C4 alkyl piperazinyl, fluoro or amino sulfonyl; or C3-C5 alkinyl; pyridyl, thiazolyl, C1-C4 alkyl oxazolyl, isoxazolyl, pyrazinyl, pyrimidyl, C1-C4 alkyl pyrazolyl, 1,2-dihydro-indanyl, phenyl or C4-C6 cycloalkyl;
pyrrolidinyl;
piperazinyl, which is unsubstituted or substituted by C1-C4 alkyl, di(C1-C4 alkyl)-amino C1-C4 alkyl, C1-C4 alkyl carbonyl, C1-C4 alkyl piperidinyl, oxo or pyrimidyl;
piperidinyl, which is unsubstituted or substituted by phenyl, pyrrolidinyl, amino carbonyl or C1-C4 alkoxy carbonyl;
morpholinyl;
tetrahydrothiazolyl; or
4-C1-C4 alkyl-1,4-diaza-cycloheptane.

9. A pharmaceutical preparation comprising a dihydroimidazole of formula rac-(I) according to claim 1, or a tautomer thereof, or a pharmaceutically acceptable salt of such a dihydroimidazole or its tautomer, and at least one pharmaceutically acceptable carrier.

10. A dihydroimidazole of formula rac-(I) according to claim 1, or a tautomer thereof, or a pharmaceutically acceptable salt of such a dihydroimidazole or its tautomer, for use in the treatment of an animal, preferably a warm-blooded animal, especially a human, that suffers from a proliferative disease.

11. The use of a dihydroimidazole of formula rac-(l) according to claim 1, or a tautomer thereof, or a pharmaceutically acceptable salt of such a dihydroimidazole or its tautomer, in the manufacture of a medicament for the treatment of a proliferative diseases.

12. A process for the manufacture of a dihydroimidazole of formula rac-(I) according to claim 1, wherein m is 0 and the other symbols and radicals have the meanings as defined for a compound of formula I in claim 1, wherein a compound of formula rac-(II) 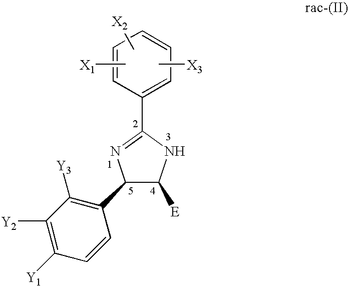
wherein the symbols and radicals have the meanings as defined in claim 1 for a compound of formula rac-(I), is reacted in a first step with a suitable reagent to replace the hydrogen at the ring nitrogen by a protection group PG and in a second step after deprotonation with a strong base with carbon dioxide to provide a carboxylic acid of formula rac-(III) 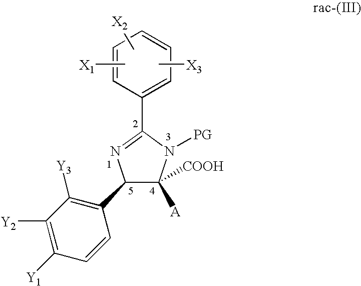
wherein the symbols and radicals have the meanings as defined for a compound of formula rac-(I) and splitting off the protection group PG under suitable reaction conditions;
where the above starting compounds of formula rac-(II) and rac-(III) may also be present with further functional groups in protected form if necessary and/or in the form of salts, provided a salt-forming group is present and the reaction in salt form is possible;
any additional protecting groups in a protected derivative of a compound of the formula rac-(I) are removed;
and, if so desired, an obtainable compound of formula rac-(I) is converted into another compound of formula rac-(I), a free compound of formula rac-(I) is converted into a salt, an obtainable salt of a compound of formula rac-(I) is converted into the free compound or another salt, and/or a mixture of isomeric compounds of formula rac-(I) is separated into the individual isomers.
